(19) United States
(12) Patent Application Publication
(10) Pub. No.: US 2026/0069171 A1
(43) Pub. Date: Mar. 12, 2026

(54) DEVICE FOR INTERMITTENT MONITORING OF A BIOMOLECULE AND METHODS OF USE THEREOF

(71) Applicant: Level Zero Health Inc., Dover, DE (US)

(72) Inventors: Yiran Jia, London (GB); Harvey Dylan Cumming, London (GB); Georgia Kate Smith, London (GB); Chapman Ho, London (GB); Andrea Eli Luigi Attipoe, London (GB); Ming Kiu Tsang, Harrow (GB); Ulviyya Rustamova, London (GB)

(21) Appl. No.: 19/326,392

(22) Filed: Sep. 11, 2025

Related U.S. Application Data
(60) Provisional application No. 63/693,265, filed on Sep. 11, 2024.

(51) Int. Cl. A61B 5/145; A61B 5/00; A61B 5/1455; A61B 5/1473; A61B 10/00

(52) U.S. Cl. CPC A61B 5/14514; A61B 5/1455; A61B 5/14735; A61B 5/6833; A61B 5/685; A61B 10/0045; A61B 2010/008

(57) ABSTRACT

A device for intermittent monitoring of a biomolecule in a human body, and use thereof. The device includes a device casing, a microneedle array including an array of needles configured to extract interstitial fluid from a user, a microfluidic system defining a fluidic channel including a biomolecule debonding agent, wherein the fluidic channel is in fluidic communication with the microneedle array and configured to facilitate flow of the extracted interstitial fluid therethrough, and an assay system in fluidic communication with the microfluidic system. The assay system includes an affinity binding agent capable of generating an indication of a level of one or more biomolecules present in the extracted interstitial fluid. The microneedle array, the microfluidic system, and assay system are disposed within the device casing.

### CROSS-REFERENCE TO RELATED APPLICATIONS

This application claims priority to, and the benefit of, U.S. Prov. No. 63/693,265, filed Sep. 11, 2024, the entirety of which is incorporated herein by reference.

### TECHNICAL FIELD

The present disclosure relates to biomolecule devices and methods of biomolecule monitoring. In particular, the present disclosure relates to devices and methods of intermittent monitoring of a single or a plurality of biomolecules.

### BACKGROUND

The human body includes many types of biomolecules, such as hormones. Hormones serve as an intricate traffic system within the body, influencing a myriad of functions from growth and development, to mood regulation, metabolism and reproductive health in both men and women. Despite the widely accepted importance of hormones to human biology, the ability to measure them properly has not kept pace with the growing significance in clinical medicine and research.

### SUMMARY

In an aspect, embodiments relate to a device for intermittent monitoring of a biomolecule in a human body. The device includes a device casing, and a microneedle array including an array of needles configured to extract interstitial fluid from a user. The device includes a microfluidic system defining a fluidic channel including a biomolecule debonding agent and in fluidic communication with a microneedle array, and configured to facilitate flow of extracted interstitial fluid therethrough. The device includes an assay system in fluidic communication with a microfluidic system. The assay system includes an affinity binding agent capable of generating a quantitative indication of a level of one or more biomolecules present in the extracted interstitial fluid. The microneedle array, microfluidic system, and assay system are disposed within the device casing.

One or more of the following features may be included. The device may include a pressure actuator configured to provide a pressure to the microfluidic system. The biomolecule debonding agent may be disposed in a coating and/or a blister pack. The assay system may be removably couplable to the device casing.

Each needle of the array of needles may have a length between about 300 μm to about 1500 μm. A tip of each needle of the array of needles may be angled at an angle of about 10 degrees to about 60 degrees. Each needle of the microneedle array may spaced apart by about 500 μm to about 5,000 μm. The microneedle array may include a rib designed to create positive pressure on a surface of skin of the user; the positive pressure may be from a range of about 10 kPA to about 100 kPa.

The microfluidic channel may include a metering zone. The microfluidic system may include a flexible membrane for inducing flow of the extracted interstitial fluid. The microfluidic system may include a fluid actuator for controlling flow of the extracted interstitial fluid.

The flow of the extracted interstitial fluid may occur through capillary pressure induced by a surface of the fluidic channel.

The assay system may include a lateral flow assay.

The affinity binding agent may be disposed in a porous strip.

The device may include an adhesive surface designed to adhere the device to a portion of the user.

At least one microneedle of the microneedle array may define two or more holes.

The biomolecule debonding agent may be a chemical, enzymatic, and/or biological agent.

In another aspect, embodiments relate to a biomolecule detection kit. The biomolecule detection kit includes a device having a device casing. A device also includes a microneedle array including an array of needles configured to extract interstitial fluid from a user. The device includes a microfluidic system defining a fluidic channel and including a biomolecule debonding agent. The fluidic channel is in fluidic communication with a microneedle array and is configured to facilitate flow of extracted interstitial fluid therethrough. The device includes an assay system in fluidic communication with the microfluidic system. The assay system includes an affinity binding agent capable of generating an indication of a level of one or more biomolecules present in extracted interstitial fluid. The microneedle array, microfluidic system, and assay system are disposed within the device casing. The biomolecule detection kit also includes a detection system configured to detect biomolecule levels based on the indication.

One or more of the following features may be included. The detection system may include a camera for capturing an image of the assay system for colorimetric analysis. The detection system may be configured to detect at least one of fluorescence and chemiluminescence of the assay system.

In yet another aspect, embodiments relate to a method of intermittent monitoring of a biomolecule in a human body using a microfluidic device. The method includes obtaining, from a microneedle array of the microfluidic device, interstitial fluid of a user. The interstitial fluid is directed, through a fluidic channel of a microfluidic system of the microfluidic device, to an assay system of the microfluidic device. An indication of a level of one or more biomolecules present in the extracted interstitial fluid is generated through the assay system.

In still another aspect, an embodiment relates to a device for intermittent monitoring of a biomolecule in a human body. The device includes a device casing, and a microneedle array including an array of needles configured to extract interstitial fluid from a user. The device also includes a microfluidic system defining a fluidic channel in fluidic communication with the microneedle array and configured to facilitate flow of the extracted interstitial fluid therethrough. The device also includes an assay system in fluidic communication with the microfluidic system, the assay system including an affinity binding agent capable of generating a quantitative indication of a level of one or more biomolecules present in the extracted interstitial fluid; and a slider mechanism configured to fluidically couple the assay system with the microfluidic system, thereby allowing the interstitial fluid to flow into the assay system. The microneedle array, the microfluidic system, and assay system are disposed within the device casing.

### BRIEF DESCRIPTION OF THE DRAWINGS

FIG. 1 is a top perspective view of a device for intermittent monitoring of a biomolecule, in accordance with an embodiment;

FIG. 2 is a top view of the device of FIG. 1;

FIG. 3 is a perspective bottom view of the device of FIG. 1;

FIG. 4 is a bottom view of the device of FIG. 1;

FIG. 5 is a side view of the device of FIG. 1;

FIG. 6 is another side view of the device of FIG. 1;

FIG. 7 is a top view of a microfluidic system, in accordance with an embodiment;

FIG. 8 is a top side perspective view of the microfluidic system of FIG. 7;

FIG. 9 is a bottom perspective view of a microneedle assembly in accordance with an embodiment;

FIG. 10 is an exploded view of a device for intermittent biomolecule monitoring in accordance with an embodiment;

FIG. 11 is an illustration of a device with a detection system;

FIG. 12 is an illustration of a removal of a casing of a device;

FIG. 13 is a flowchart of a method of generating an indication of a level of one or more biomolecules using a device in accordance with an embodiment;

FIG. 14 is a photograph of sensing strip readouts;

FIG. 15 is a graph of the data generated through the sensing strips of FIG. 14;

FIGS. 16A-16C are perspective views of a device, sensing strip, and computer readout;

FIG. 17A-17F are schematic views illustrating a method of use of a device in accordance with an embodiment;

FIGS. 18A-18B are perspective views of various components of a device in accordance with an embodiment;

FIG. 19A-19B are perspective views of a microfluidic chamber of a device in accordance with an embodiment;

### DETAILED DESCRIPTION

Maintaining a delicate balance of hormones is essential for overall well-being. Hormonal imbalances can severely impact an individual's quality of life and give rise to pathological conditions that may deserve medical attention. These imbalances can manifest in diverse ways, affecting energy levels, sleep patterns, cognitive function, fertility and more.

The challenge lies in capturing the volatility of hormones, which can fluctuate significantly, making diagnosis and management less effective. In healthcare today, the primary method for measuring hormones remains blood draws, providing only a snapshot of data at a specific moment in time, which falls short in capturing this dynamic nature of hormonal fluctuations.

Hormonal imbalances in women and men can lead to various health issues, affecting reproductive health, mood, and overall well-being. According to the American Thyroid Association, about 12% of women will experience thyroid disorders in their lifetime, often resulting in hormonal imbalances. Polycystic Ovary Syndrome (PCOS) affects around 10% of women of childbearing age, causing irregular periods and elevated androgen levels. 70% of these women remain undiagnosed. Additionally, menopause, occurring around age 51 on average, involves a significant hormonal shift, impacting various aspects of health, including bone density and cardiovascular health. These statistics underscore the prevalence and impact of hormonal imbalances on women's health, necessitating awareness and appropriate medical attention.

Hormonal fluctuations play a crucial role in various aspects of health, yet monitoring these changes continuously or even frequently poses challenges. One of the primary limitations lies in the variability of biomolecule levels throughout the day, week, or month. Biomolecule levels fluctuate in response to various factors such as stress, diet, exercise, and the menstrual cycle.

Disclosed herein are devices and methods for intermittent monitoring of biomolecules in the human body using minimally invasive technology. The devices include a microneedle array for extracting interstitial fluid, a microfluidic system for processing the extracted fluid including debonding agents to separate biomolecules from binding proteins, and an assay system for detecting and quantifying biomolecule levels. The devices may be designed to provide near real-time biomolecule monitoring capabilities that overcome the limitations of traditional blood-based testing methods, that provide only snapshots of biomolecule levels at specific moments in time. The technology addresses the critical need for continuous or frequent biomolecule monitoring to capture the dynamic nature of hormonal fluctuations that occur throughout daily, weekly, and monthly cycles, particularly for reproductive health monitoring, metabolic assessment, and endocrine disorder management.

Devices and methods described herein may allow for extraction of interstitial fluid. Interstitial fluid may contain one or more biomolecules of an individual. Biomolecules may include, but are not limited to, hormones, peptide hormones, small molecules, metabolites, proteins, enzymes, neurotransmitters, cytokines, or other biomolecules present in interstitial fluid. Biomolecule concentrations in interstitial fluid may range from about 0.1 pmol/L to about 1000 nmol/L or greater, depending on the specific biomolecule being monitored and physiological conditions of the user, with equivalent mass-based concentrations ranging from about 0.1 ng/dL to about 10,000 ng/dL or greater. Monitoring of biomolecules within interstitial fluid of a user may allow for less invasive methods of tracing levels of hormones in an individual compared to venous blood draws or other invasive methods. Compositions of interstitial fluid may correlate with blood compositions, allowing for an accurate representation of hormone levels to be discerned. For instance, devices relying on interstitial fluid monitoring may be as accurate as Continuous Glucose Monitoring (CGM) devices, despite being less invasive.

In some embodiments, angled tips of microneedles may provide advantages in optimizing a low-force insertion into skin of a patient and/or minimizing pain associated therewith. The angled configuration of microneedle tips, ranging from about 10 degrees to about 60 degrees as described herein, may minimize the force required for skin penetration while maintaining structural integrity of the microneedles. This reduced insertion force may enhance user comfort during device application and may improve the reliability of microneedle deployment across varying skin types and conditions.

In some embodiments, length range of about 300 μm to about 1500 μm for each microneedle of the array provides the advantage of effectively capturing interstitial fluid in the dermis layer. Effectively capturing interstitial fluid may include drawing amounts of interstitial fluid at rates of about 0.01 μL/min to about 100 μL/min. This penetration depth range may ensure that the microneedles reach the interstitial fluid-rich dermal layers while avoiding deeper penetration that could cause discomfort or reach pain-sensitive nerve endings and/or avoid drawing blood, thereby optimizing both fluid extraction efficiency and user comfort during biomolecule monitoring.

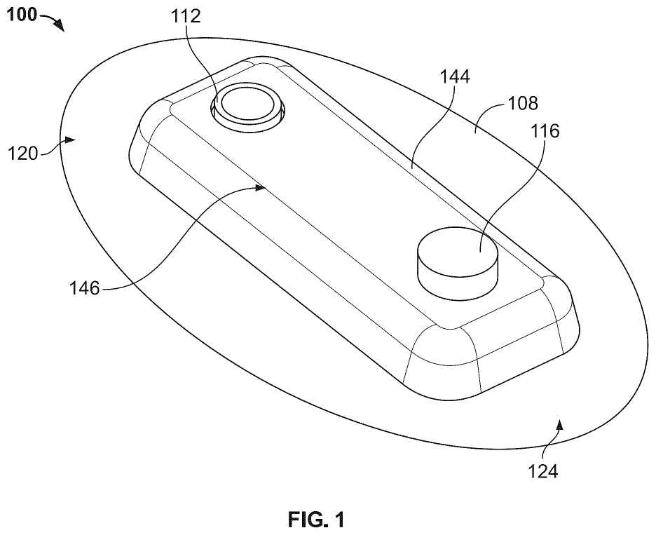

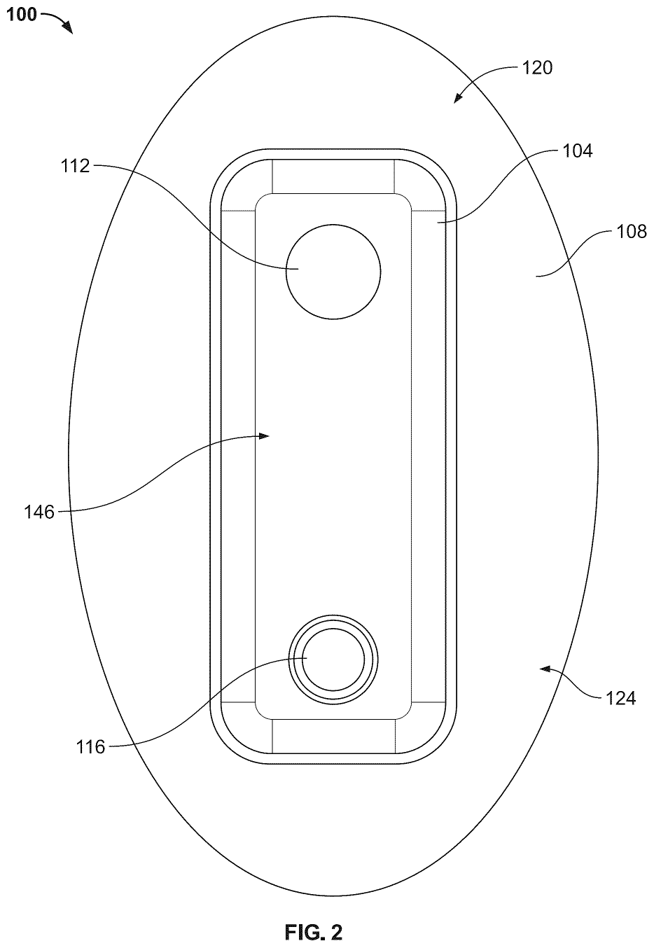

Referring to FIGS. 1 and 2, a device 100 for intermittent monitoring of biomolecule may include a device casing 144, also referred to herein as a housing. Device casing 144 may be, but is not limited to, rectangular, oval, circular, triangular or other shapes. In some embodiments, device casing 144 may be rectangular with curved edges. Device casing 144 may have a width of about, but not limited to, 10 mm to about 150 mm or greater. Device casing 144 may have a length of about, but not limited to, 10 mm to about 150 mm or greater. In some embodiments, a length of device casing 144 may be greater than a width of device casing 144, or vice versa. Device casing 144 may have tapered edges. For instance, a top surface 146 may extend into a bottom surface of device casing 144 at an angle. Angles between an edge of top surface 146 and an edge of a bottom surface of device casing 144 may be between about, but not limited to, 0 degrees to about 15 degrees or greater. For instance, a width of device casing 144 at a bottom surface may be greater than a width of device casing 144 at top surface 146. In some embodiments, device casing 144 may have a height of about, but not limited to, 1 mm to about 100 mm or greater. In some embodiments, device 100 may include a heat applier configured to warm a skin surface of a user. A heat applier may be a resistive, radiative, laser, or other form of heat applier. A heat applier may be positioned within device casing 144 or integrated with a bottom surface of device casing 144. A heat applier may include heating elements such as, but not limited to, resistive heating elements, thermoelectric heating elements, chemical heating pads, and/or infrared heating elements. A heat applier may be configured to generate temperatures ranging from about 1° C. to about 50° C. or greater, without limitation. Ranges of heat between about 1° C. to about 50° C. or greater may enhance biomolecule diffusion from blood plasma to interstitial fluid without causing thermal discomfort or tissue damage. A heat applier may be activated manually through user input or automatically upon device deployment, and may include temperature control mechanisms to maintain consistent heating within safe operating ranges. In some embodiments, instead of an integrated heat applier within device 100, a separate heat applier may be used that is positioned external to device casing 144 and applied to the skin surface prior to or during device operation.

Device casing 144 may be made out of material such as, but not limited to, plastic, polymers, or other materials. In some embodiments, device casing 144 may be made of a rigid material, such as but not limited to a rigid plastic. Rigid plastics may include, but are not limited to, polycarbonate, acrylonitrile butadiene styrene (ABS), polyoxymethylene (POM), polyetheretherketone (PEEK), polyphenylene oxide (PPO), and other rigid polymers. In some embodiments, device casing 144 may have a hollow interior. One or more components of the device 100 may be disposed within the hollow interior of device casing 144. Device casing 144 may provide protection for the components of device 100 from one or more environmental conditions. Environmental conditions may include, but are not limited to, air, dust, water, humidity, light, ultraviolet light, pressures, or other environmental conditions. For instance and without limitation, device casing 144 may seal components of device 100 from external liquid sources.

Device 100 may include a pressure actuator 112. Pressure actuator 112 may be positioned at proximal end 120 of device 100. In some embodiments, pressure actuator 112 may be a circular or other shaped button positioned at a center of proximal end 120 of device 100. Pressure actuator 112 may be depressible. For instance, pressure actuator 112 may receive kinetic force from a user in the form of a pressing motion in a direction downwards towards a bottom surface of housing 104. Pressure actuator 112 may move vertically downwards along with a user's finger, thumb, or other appendage. In some embodiments, pressure actuator 112 may travel between about 0.1 inches to about 1 inch or greater in a vertical orientation with respect to a vertical axis of device 100. Pressure actuator 112 may apply a form of pressure to a portion of a user at a bottom side of device 100. For instance, pressure actuator 112 may be in mechanical communication with a mechanical component such as, but not limited to, a rib, fin, or other mechanical component. A movement of pressure actuator 112 may cause a movement of a mechanical component, which may apply a pressure to a portion of a microfluidic system within device 100. A pressure applied to a portion of a microfluidic system within device 100 may facilitate movement of one or more fluids within the microfluidic system.

Device 100 may include a needle inserter 116. Needle inserter 116 may be a circular or other shaped element that may be positioned at a center of distal end 124 of device 100. For instance, needle inserter 116 may be positioned equidistant between sides of device 100 at distal end 124. In some embodiments, a center of needle inserter 116 may be aligned with a longitudinal axis of device 100. A longitudinal axis of device 100 may extend in a direction from proximal end 120 towards distal end 124 of device 100. In some embodiments, both pressure actuator 112 and needle inserter 116 may be aligned with a longitudinal axis of device 100 such that centers of pressure actuator 112 and needle inserter 116 are aligned. In other embodiments, either or both of pressure actuator 112 and needle inserter 116 may be off-center with respect to a longitudinal axis of device 100. A length between needle inserter 116 and pressure actuator 112 may be about, but is not limited to, 10 mm to about 200 mm or greater.

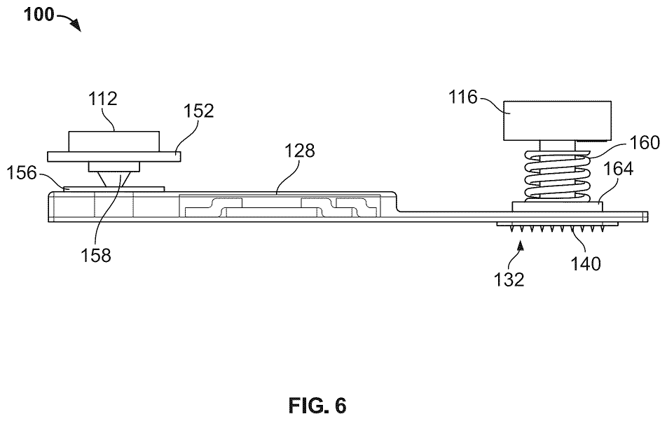

Needle inserter 116 may be mechanically coupled to a microneedle array. For instance, kinetic energy received at needle inserter 116 in a downwards direction towards a bottom of device 100 may cause deployment of a microneedle array. Needle inserter 116 may receive kinetic energy from a user, such as through a finger, thumb, hand, or other body part of a user. A deployment of a microneedle array may cause one or more microneedles of the microneedle array to pierce a skin of a user. Operations of pressure actuator 112 and needle inserter 116 are described in greater detail below with reference to FIG. 6.

Device casing 144 may be coupled to an adhesive patch 108. Adhesive patch 108 may be circular, oval, or other shapes. In some embodiments, adhesive patch 108 may have a perimeter greater than a length and/or width of device casing 144. For instance and without limitation, adhesive patch 108 may have a length of about 100 mm to about 300 mm or greater. Adhesive patch 108 may have a width of about 100 mm to about 300 mm or greater. In some embodiments, adhesive patch 108 may have an adhesive on a top surface. A top surface of adhesive patch 108 may be a surface of adhesive patch 108 that may contact a bottom side of device casing 144. Adhesives may include, but are not limited to, acrylic adhesives, silicone adhesives, hydrocolloid adhesives, polyurethane adhesives, or other biocompatible adhesives. Device casing 144 may be coupled to adhesive patch 108 at a top surface of adhesive patch 108 using a first adhesive. A first adhesive may have an adhesive strength of about 0.1 N/mm to about 5.0 N/mm or greater. Adhesive patch 108 may be coupled to a user at a bottom surface of adhesive patch 108 using a second adhesive. A second adhesive may be designed to adhere to skin and may have an adhesive strength of about 0.01 N/mm to about 1.0 N/mm or greater to ensure secure attachment while allowing for comfortable removal. A first adhesive and second adhesive may be the same type of adhesive or different types of adhesives. For instance, a first adhesive may be a stronger acrylic adhesive for securing device casing 144 to adhesive patch 108, while a second adhesive may be a gentler silicone or hydrocolloid adhesive. A silicone or hydrocolloid adhesive may allow for minimization of skin irritation and/or may allow for less force required for removal of adhesive patch 108. Adhesive patch 108 may be couplable to, but not limited to, a user's arm, back, leg, torso, shoulder, or any other part of a user, with the adhesive patch 108 adhering to the bottom edge of device casing 144 and providing additional adhesive area around the perimeter while excluding the microneedle array area to allow for needle deployment. Adhesive patch 108 may be removable through a force applied in a direction away from and/or towards a side of a skin of a user. For instance, a user may pull an edge of adhesive patch 108 which may overcome an adhesive bonding strength of a bottom surface of adhesive patch 108 and the user's skin, allowing for removal of adhesive patch 108 and/or device 100 from the user.

In some embodiments, device 100 may be symmetrical across a longitudinal axis of device 100. For instance, pressure actuator 112 and needle inserter 116 may be positioned along a longitudinal axis of device 100 such that device 100 is symmetrical across a longitudinal axis of device 100. Device casing 144 may be positioned at a center of adhesive patch 108. Positioning of device casing 144 at a center of adhesive patch 108 may allow for balanced weight distribution and/or balanced adhesion of device 100 to a user. Device 100 may have a weight of between about 50 grams to about 200 grams or greater.

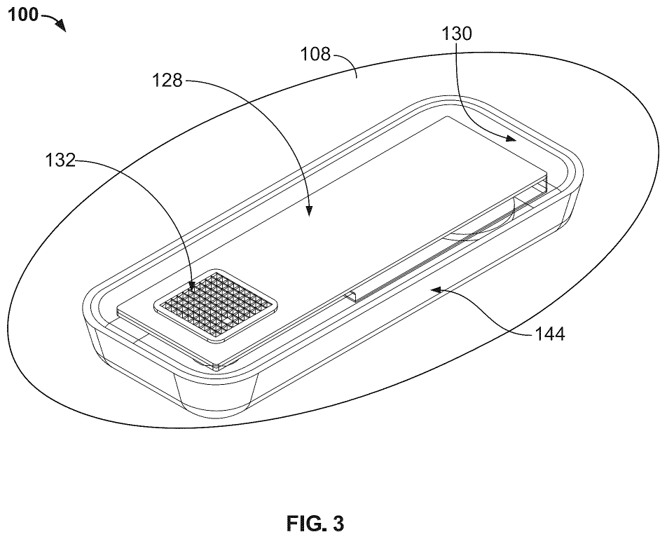

Referring now to FIG. 3, device casing 144 may define a cavity 130. Cavity 130 may be an interior of device casing 144 that may be hollow. For instance, a microfluidic system 128 may be positioned within cavity 130 of device casing 144. Microfluidic system 128 may be in fluidic communication with a microneedle array 132. Microneedle array 132 may be positioned beneath microneedle inserter 116 and may be mechanically coupled to microneedle inserter 116. In some embodiments, microneedle array 132 may have a width of about 200 μm to about 1,000 μm and/or a length of about 500 μm to about 1,500 μm, without limitation. Microneedle array 132 may have dimensions of about, but not limited to, 200 μm by 1,000 μm or greater. Microneedle array 132 may be an arrangement of two or more microneedles. Microneedle array 132 may include an array of pyramid or other shaped, hollow microneedles fabricated from a polymer-based, silicone, stainless steel, or other material. In some embodiments, microneedle array 132 may include an array of between about 5×5 to about 50×50 microneedles or greater than about 50×50 microneedles. A spacing between microneedles of microneedle array 132 may be about, but is not limited to, 100 μm to about 5,000 μm or greater. Each microneedle of microneedle array 132 may have a base diameter of about, but not limited to, 100 μm to about 1,500 μm. Microneedles of microneedle array 132 may be pointed and hollow structures that may have a length of about, but not limited to, 100 μm to about 1,500 μm. Each microneedle of microneedle array 132 may include a lumen with a diameter between about, but not limited to, 50 microns to about 300 microns. Lumen diameters below about 50 μm may increase susceptibility to clogging by skin debris, while diameters above about 300 μm may complicate penetration mechanics and hinder stable interstitial fluid withdrawal by disrupting capillary continuity and creating non-uniform flow. In some embodiments, each microneedle may include two fluidic apertures that are diametrically opposed and offset from a central longitudinal axis of the microneedle. This configuration may preserve sharpness of the microneedle tip while enabling fluid access along the needle shaft rather than solely at the tip. The offset distance may be optimized so that the apertures reach interstitial fluid-rich dermal layers during skin penetration. In some embodiments, each microneedle may include two or more lumens or holes. Multiple lumens and/or holes of each microneedle may enhance fluid extraction capabilities and provide redundant fluid pathways. Each microneedle of microneedle array 132 may be designed to puncture skin of a user. In alternative embodiments, microneedle array 132 may include solid microneedles configured to create microchannels in the skin through which interstitial fluid may be extracted using negative pressure or suction mechanisms. Solid microneedles may be fabricated using micro machining processes including computer numerical control (CNC) machining, injection molding, chemical etching, or additive manufacturing techniques. Microneedles may be shaped as pyramids, cylinders, cones, or obelisks, with protruding lengths ranging from about 100 μm to about 1,500 μm. Solid microneedles may be fabricated from silicon, metals including stainless steel or titanium, or polymer materials, and may be designed to work in conjunction with suction systems that apply negative pressure to extract interstitial fluid through the created microchannels. For instance, each microneedle of microneedle array 132 may be designed to puncture depths of skin of a user of between about, but not limited to, 100 μm to about 1,500 μm.

A depth of about 100 μm to about 1,500 μm may cause one or more microneedles of microneedle array 132 to contact fluid of a user. Fluid of a user may be, but is not limited to, interstitial fluid, blood, or other fluids. Interstitial fluid is a clear fluid that surrounds cells in tissues and may contain biomolecules and other biomolecules that have diffused from blood plasma. Each microneedle of microneedle array 132 may be designed to puncture depths of skin of a user to access fluid of a user and direct it into microfluidic system 128 through a corresponding lumen. In some embodiments, fluid may be directed into each microneedle of microneedle array 132 through fluid pressure of the human body. For instance, pressures experienced by one or more microneedles of microneedle array 132 may be between about, but not limited to, 1 kPa to about 100 kPa or greater. In some embodiments, fluid may flow into each microneedle of microneedle array 132 at a rate of about, but not limited to, 0.1 μL/min to about 10 μL/min or greater. Each needle of microneedle array 132 may be hollow, allowing for fluid extraction through internal lumens that extend from the needle tip to the base of the needle structure.

In some embodiments, a lumen of a microneedle may be offset from a central longitudinal axis of microneedle array 132 by between about 0 to about 500 μm. An offsetting of a lumen of a microneedle with respect to a central longitudinal axis of microneedle array 132 may allow for improved fluid collection by positioning the lumen away from a center of a microneedle tip, which may prevent blockage that may occur if the lumen were centered.

In some embodiments, an offsetting of a lumen of a microneedle with respect to a central longitudinal axis of microneedle array 132 may allow for capillary-driven transport of fluid through the microneedle. Capillary driven transport may include a movement of fluid through narrow channels or lumens without the need for external pumping mechanisms. Capillary driven transport may occur due to surface tension forces and an interaction between fluid and channel walls of a microneedle, which may drive fluid into the microneedle. In microneedle array 132, each microneedle may utilize capillary action to draw fluid through its lumen and into microfluidic system 128. In some embodiments, each microneedle of microneedle array 132 may include a hydrophilic surface. Hydrophilic surfaces may be materials and/or surfaces that may attract liquid molecules, such as water, through hydrogen bonding and/or other intermolecular forces. In some embodiments, a contact angle of a channel wall of a microneedle may be less than about 90 degrees, which may increase liquid travel within the microneedle. In some embodiments, each microneedle may include or be coated in a hydrophilic material such as, but not limited to, polyethylene glycol, polyvinyl alcohol, hydrogels, or other hydrophilic materials.

In some embodiments, a tip angle of each microneedle may be between about 10 degrees to about 60 degrees. A tip angle of each microneedle of greater than about 10 degrees may allow for increased skin penetration relative to an angled tip having an angle of less than about 10 degrees. In some embodiments, a tip angle of each microneedle may increase skin penetration while keeping a structural integrity of each microneedle. For instance, tip angles of over about 60 degrees may cause microneedles to become fragile, while lower tip angles may provide more material at a tip of a microneedle, enhancing structural integrity of the microneedle.

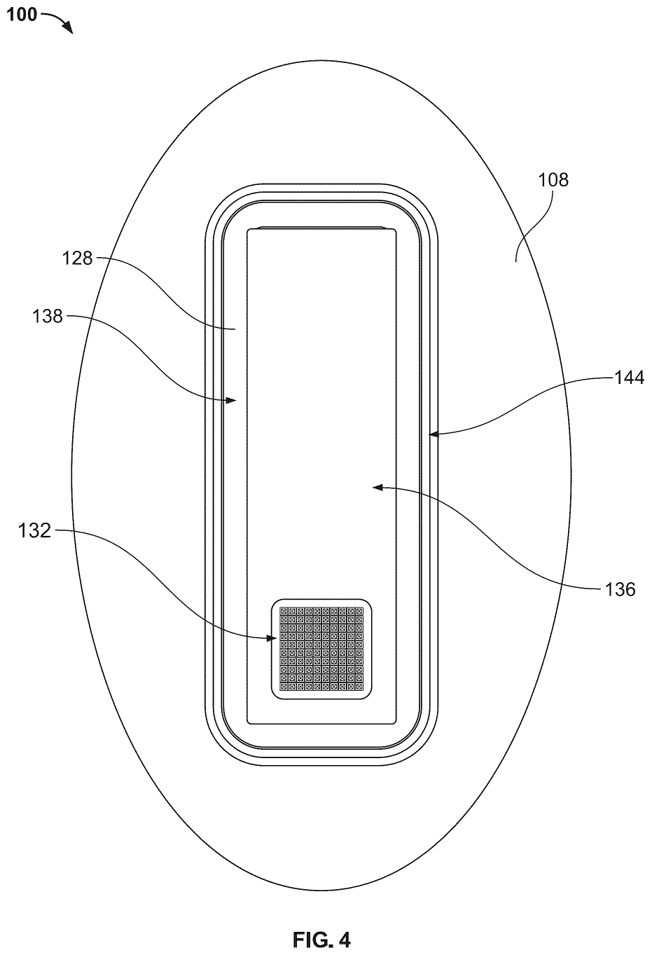

Referring to FIG. 4, device casing 144, in some embodiments, adhesive patch 108 may have an aperture sized according to the dimensions of device casing 144. For instance, an aperture of adhesive patch 108 may allow for bottom surface 138 of device casing 144 to pass through adhesive patch 108 and contact a portion of a user. In some embodiments, an aperture of adhesive patch 108 may allow for an entirety of bottom surface 138 of device casing 144 to pass therethrough. In other embodiments, adhesive patch 108 may have an aperture sized for microneedle array 132. An aperture sized according to the dimensions of microneedle array 132 may allow one or more microneedles of microneedle array 132 to contact a portion of a user while avoiding adhesive patch 108. In some embodiments, bottom surface 138 may have padding 136. Padding 136 may be made of materials such as, but not limited to, silicone, polyurethane foam, hydrogel, or other soft elastomeric materials. Padding 136 may be positioned around a perimeter of microneedle array 132 on bottom surface 138 of device casing 144. Padding 136 may have a thickness of about 0.5 mm to about 5 mm or greater. In some embodiments, padding 136 may be circular, oval, or rectangular in shape and may have dimensions that extend about 2 mm to about 10 mm beyond an outer perimeter of microneedle array 132. Padding 136 may be designed to conform to contours of a user's skin, providing improved contact of bottom surface 138 with a user's body part and/or adhesive pad. Padding 136 may distribute pressure evenly around microneedle array 132, which may reduce discomfort and improve adhesion of device 100 to the user. In some embodiments, padding 136 may have a Shore A hardness of about, but not limited to, 10 to about 50, providing softness for user comfort while maintaining structural support for microneedle array 132.

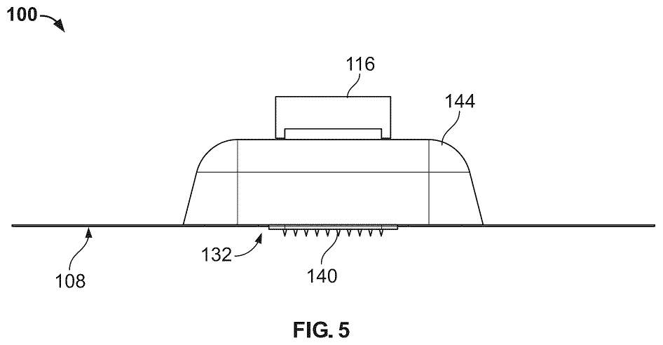

Referring now to FIGS. 5 and 6, in use, a user may press downwards on needle inserter 116, such as in a direction towards adhesive patch 108. Forces required to move needle inserter 116 may be between about, but not limited to, 1 N to about 50 N or greater. A downwards movement of needle inserter 116 may cause deployment of microneedle array 132. For instance, microneedle array 132 may travel vertically by about, but not limited to, 1 mm to about 10 mm or greater. A vertical travel of microneedle array 132 may cause one or more microneedles 140 to pierce a skin of a user. One or more microneedles 140 may travel into a user at depths of, but not limited to, 1 mm to about 10 mm or greater. Insertion of one or more microneedles 140 may cause one or more microneedles 140 to contact fluid within a user. Fluid may be, but is not limited to, blood, interstitial fluid, or other fluids of the human body. In some embodiments, fluid may travel into one or more lumens of one or more microneedles 140. Fluid may be driven through bodily pressure and/or capillary action into one or more microneedles 140.

Device 100 may be designed for single-use deployment of microneedle array 132. In some embodiments, once needle inserter 116 is depressed and microneedle array 132 is deployed, microneedles 140 may be held in an extended position for a predetermined duration through one or more locking mechanisms. Locking mechanisms may include, but are not limited to, latches, hooks, or other locking mechanisms. In some embodiments, needle inserter 116 may include a spring that may expand, allowing microneedles 140 to remain in an extended position while the spring is expanded.

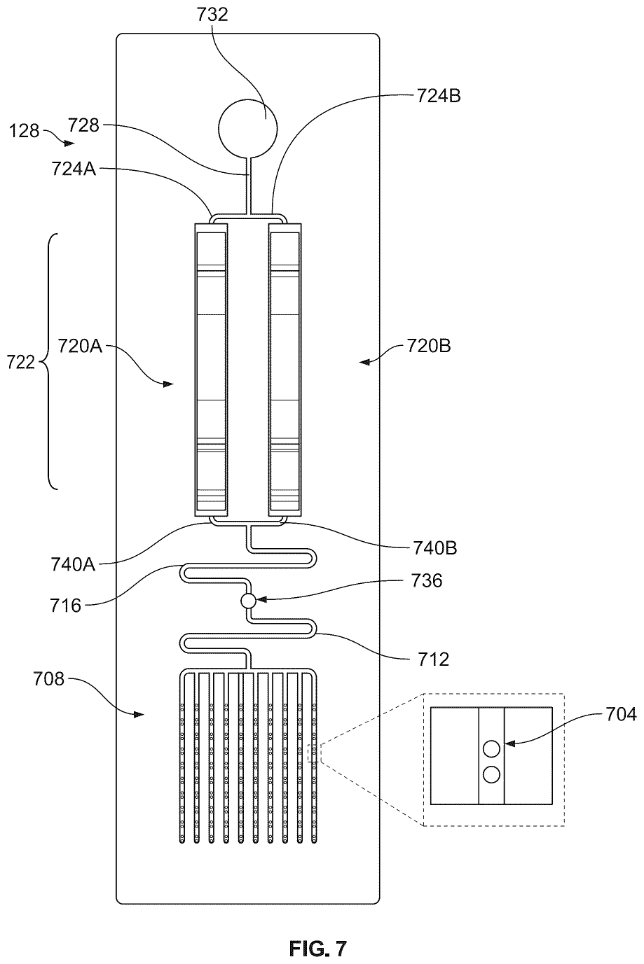

Microfluidic system 128 may include one or more fluidic and/or microfluidic channels that may allow for travel of fluid from microneedles 140 to an assay system, described in more detail below with reference to FIG. 7.

Pressure actuator 112 may be mechanically coupled to pressure applier 158. Pressure applier 158 may be a conical, rectangular, oval, or other shaped rigid structure. In some embodiments, pressure applier 158 is part of pressure actuator 112. Pressure actuator 112 may include overmold 152. Overmold 152 may create a sealed enclosure around microfluidic system 128. Pressure actuator 112 may include pressure membrane 156. Pressure membrane 156 may be a flexible elastomeric material such as silicone, polyurethane, or other polymer. Pressure membrane 156 may be configured to create negative pressure in one or more channels of microfluidic system 128 when stretched by pressure applier 158. Alternatively, microfluidic system 128 may include a fluid actuator for controlling flow of extracted interstitial fluid, providing active pressure control ranging from about, but not limited to, −10 kPa to about −100 kPa. A user may press on pressure actuator 112, which may cause pressure applier 158 to move in a downwards motion towards a bottom of device 100. A movement of pressure applier 158 in a downwards motion towards a bottom of device 100 may cause pressure membrane 156 to stretch. A stretching of pressure membrane 156 may create negative pressure within microfluidic system 128. Negative pressure may be pressure that is less than that of atmospheric pressure, such as less than about 100 kPa. For instance, negative pressure generated by a movement of pressure membrane 156 may range from about, but not limited to, −1 kPa to about −20 kPa. In alternative embodiments, microfluidic system 128 may employ active negative pressure systems including fluid actuators that generate negative pressure ranging from about −10 kPa to about −100 kPa. Fluid actuator-based systems may be connected via microfluidic connectors to one or more microfluidic channels and may provide more precise pressure control compared to membrane-based systems. In some embodiments, microfluidic system 128 may include one or more valves. In embodiments where microfluidic system 128 includes one or more valves, pressure may be regulated by the one or more valves and may reach values of about, but not limited to, −10 kPa to about −50 kPa. Pressure membrane 156 may work in conjunction with pressure applier 158 to regulate fluid flow through microfluidic system 128 by creating vacuum conditions that draw fluid from microneedles 140 into fluidic channels of microfluidic system 128.

Needle inserter 116 may include spring 160. Spring 160 may be wrapped around a base portion of needle inserter 116. Spring 160 may be wrapped in a clockwise or counter clockwise direction. In some embodiments, spring 160 may have a spring constant of about 1 N/mm to about 50 N/mm. Spring 160 may have a spring constant greater than about 1 N/mm, which may aid in preventing accidental deployment of microneedle array 132. Spring 160 may be made of materials such as stainless steel, titanium, or other spring materials that maintain their elastic properties over repeated use. In some embodiments, spring 160 may be a compression spring, helical compression spring, conical spring, wave spring, or other type of spring. Spring 160 may be configured to provide resistance against downward movement of needle inserter 116. For instance, spring 160 may resist kinetic energy received from user input until a spring constant of spring 160 is overcome. In some embodiments, when a user applies downward force to needle inserter 116, spring 160 may compress. Spring 160 may be mechanically coupled to plunger 164. Plunger 164 may be round, circular, or other shapes. Plunger 164 may be mechanically coupled to microneedle array 132. Plunger 164 may be fabricated from materials such as, but not limited to, rubber, stainless steel, aluminum, polymer-based materials such as polyoxymethylene (POM) or polyetheretherketone (PEEK), or other materials. In some embodiments, decompression of spring 160 may provide a force on plunger 164 in a downwards motion towards a bottom of device 100. A downwards motion of plunger 164 may cause deployment of microneedle array 132, thereby inserting one or more microneedles 140 into a portion of a user.

Referring now to FIG. 7, an embodiment of microfluidic system 128 may be in fluidic communication with microneedle fluidic outputs 704. In some embodiments, microneedle fluidic outputs 704 may be part of a lumen of a corresponding microneedle. Microneedle fluidic outputs 704 may be fluidically coupled to microneedle array fluidic channels 708 (also referred to as “fluidic channels 708”). Fluidic channels 708 may have curved walls that may allow for fluid to travel therethrough. For instance, each fluidic channel 708 may have curved walls with a lumen extending therethrough. Fluid may flow through a lumen of a fluidic channel 708 between curved walls of the fluidic channel along a length of the fluidic channel. Each microneedle fluidic output 704 may be an opening of a microneedle in which fluid may flow. In some embodiments, microneedle fluidic outputs 704 may have a diameter of about 0.1 μm to about 1.0 μm or greater, without limitation. In alternative embodiments, microneedle fluidic outputs 704 may have diameters ranging from about, but not limited to, 0.05 μm to about 10 μm, or from about 0.5 μm to about 5 μm. Microneedle fluidic outputs 704 may have circular, elliptical, rectangular, or polygonal cross-sectional shapes. In some embodiments, microneedles may be arranged in pairs of two and may be placed linearly along a corresponding fluidic channel of fluidic channels 708. Alternatively, microneedles may be arranged in groups of three, four, five, or more, or may be arranged in staggered, hexagonal, or grid patterns. A spacing between microneedle fluidic outputs 704 may be about, but is not limited to, 1 mm to about 5 mm or less than about 1 mm. In various embodiments, the spacing may range from about 0.1 mm to about 10 mm, or from about 0.5 mm to about 2 mm. A single fluidic channel of fluidic channels 708 may have 20 or more microneedle fluidic outputs 704. In alternative embodiments, a single fluidic channel may have between 1 and 100 microneedle fluidic outputs 704, without limitation. Each microneedle fluidic output 704 may be fluidically coupled to a corresponding fluidic channel 708 such that fluid received from a microneedle may flow through a corresponding microneedle fluidic output 704 and into a corresponding fluidic channel 708. A fluidic coupling may include direct connection, or connection through intermediate channels or valves. In some embodiments, microfluidic system 128 may have two or more fluidic channels 708, each with corresponding microneedle fluidic outputs 704. In some embodiments, microfluidic system 128 may have between about, but not limited to, 1 to about 20 or more fluidic channels 708. In various configurations, microfluidic system 128 may include between 2 and 100 fluidic channels 708, or between 5 and 50 fluidic channels 708, without limitation. Fluidic channels 708 may be arranged in rows. For instance, microfluidic system 128 may have between about, but not limited to, one to about 10 or more rows of fluidic channels 708. Rows may be parallel, angled, or curved relative to each other. Fluidic channels 708 may have varying cross-sectional areas along their length, may include flow restrictors or expansion chambers, and/or may be fabricated from materials including but not limited to silicon, glass, polymers, ceramics, or combinations thereof.

In some embodiments, fluidic channels 708 may fluidically merge into a first channel 712. First channel 712 may be a fluidic channel fluidically coupled to each of fluidic channels 708. For instance, first channel 712 may include one or more curved walls and a lumen extending therethrough. Fluidic channel 712 may receive fluid from one or more of fluidic channels 708. In some embodiments, fluidic channel 712 may aggregate amounts of fluid from fluidic channels 708 into a singular stream of fluid. For instance, ends of each of fluidic channels 708 may be fluidically connected. An end of fluidic channel 712 may be fluidically coupled to each end of fluidic channels 708. Fluid may flow from one or more fluidic channels 708 into first channel 712. Fluid may flow into first channel 712 at rates of about, but not limited to, 1 μL/min to about 100 μL/min or greater. In alternative embodiments, flow rates may range from about 0.1 μL/min to about 500 μL/min, or from about 0.01 μL/min to about 10 μL/min. The merging configuration may include sequential merging where fluidic channels 708 merge one at a time into first channel 712, simultaneous merging where multiple channels merge at a single junction, or hierarchical merging where channels first merge into intermediate channels before merging into first channel 712. First channel 712 may include flow control elements such as valves, fluid actuators, flow restrictors, or pressure regulators to control fluid aggregation. The channel may have a variable cross-sectional area, with expansion or contraction regions to facilitate mixing or flow control. In some embodiments, first channel 712 may include mixing elements such as serpentine sections, obstacles, or surface textures to promote fluid homogenization. The merging junction may be Y-shaped, T-shaped, or have multiple inlet ports arranged radially or linearly. First channel 712 may be fabricated from the same or different materials as fluidic channels 708, and may include transparent sections for optical monitoring of fluid flow. The channel may incorporate sensors for monitoring flow rate, pressure, temperature, or fluid composition during aggregation.

First channel 712 may be curved and may be in a serpentine configuration with multiple interconnected curved segments and straight channel sections. For instance, first channel 712 may be serpentine shaped with alternating curved portions and linear segments that create a tortuous flow path. The serpentine geometry may include between 2 to 20 curved segments, with each curved segment having a radius of curvature ranging from about, but not limited to, 0.1 mm to about 5 mm. In some embodiments, first channel 712 may act as a metering zone that provides precise volumetric control of fluid flow. In some embodiments, first channel 712 may be referred to as a “metering zone.” For instance, first channel 712 may act to regulate a flow of fluid through microfluidic system 128 by creating controlled fluidic resistance at predetermined locations. In some embodiments, first channel 712 may have one or more curves which may allow for fluid aggregation at the one or more curves, where each curve creates a localized pressure drop and temporary fluid accumulation zone. First channel 712 may have one or more straight paths which may regulate a flow of fluid between curves of first channel 712, with straight sections maintaining consistent cross-sectional areas and predictable flow characteristics. One or more curves may create areas of increased fluidic resistance that may slow fluid velocity and allow for controlled volume accumulation at each curve junction. The curved segments may have varying geometries including circular arcs, elliptical curves, or parabolic sections to optimize flow characteristics. For example and without limitation, the curves may create fluidic resistance values of about 1 Pa·s/m³ to about 100 Pa·s/m³, which may reduce fluid velocities by about 10% to about 90% compared to straight channel sections. One or more straight paths may maintain fluid velocities of about 0.1 mm/s to about 10 mm/s, while the curves may accumulate fluid volumes of about 0.1 μL to about 10 μL at each junction before allowing continued flow downstream. A serpentine configuration may provide enhanced mixing capabilities through Dean flow effects at curved sections, improved particle separation through differential flow velocities, and increased residence time for chemical reactions or biological processes. Channel width may vary from about 10 μm to about 1000 μm, with depth ranging from about 5 μm to about 500 μm, creating aspect ratios optimized for specific fluid handling requirements.

In some embodiments, straight paths of first channel 712 between curves of first channel 712 may provide predictable flow channels with defined cross-sectional areas that regulate volumetric flow rates through controlled fluidic resistance. For instance, cross-sectional areas of first channel 712 may be about, but not limited to, 0.001 mm² to about 0.05 mm² or greater. Fluidic resistance in straight path sections of first channel 712 may be between about, but not limited to, about 1 Pa·s/m³ to about 100 Pa·s/m³, In some embodiments, a fluidic resistance of straight paths of first channel 712 may be lower than a fluidic resistance of curved portions of first channel 712, which may allow for an increased fluid velocity in straight portions of first channel 712 compared to curved portions of first channel 712. Fluid velocities of straight paths of first channel 712 may be about, but are not limited to, 0.1 mm/s to about 10 mm/s. Serpentine geometry of first channel 712 may work in conjunction with capillary driven transport and pressure control systems of microfluidic system 128 to ensure precise metering of interstitial fluid volumes before delivery to downstream assay components. In some embodiments, the flow of extracted interstitial fluid may occur exclusively through capillary pressure induced by hydrophilic surface treatments of the fluidic channel walls, eliminating the need for external pumping mechanisms. In such capillary driven embodiments, fluidic channel surfaces may be treated or coated with hydrophilic materials to achieve contact angles between about 5 to about 25 degrees. Dimensions for capillary driven systems include microneedle channel inner diameters ranging between about 50 μm to about 200 μm, microfluidic channel heights between about 20 μm to about 80 μm, and/or channel widths ranging from about 100 μm to about 300 μm, without limitation.

Microfluidic system 128 may include fluidic valve 736 (also referred to as “valve 736”). Fluidic valve 736 may be a capillary valve, hydrophobic valve, or mechanical valve. Capillary valves may utilize surface tension forces to control fluid flow by creating hydrophobic barriers that prevent fluid advancement until sufficient pressure is applied. Capillary valves may operate by creating contact angles between 5 to 25 degrees on channel surfaces. Contact angles may allow for controlled fluid stopping at valve locations until pressure thresholds of about 1 kPa to about 10 kPa are exceeded. Hydrophobic valves may employ hydrophobic coatings or materials that repel aqueous fluids, allowing for controlled fluid stopping and starting based on applied pressure differentials. Hydrophobic valves may operate through gas permeable membranes or hydrophobic surface treatments that create fluid barriers. Gas permeable membranes may allow air to pass while blocking liquid flow until sufficient pressure overcomes surface tension forces. Mechanical valves may include flexible membrane valves, check valves, or pinch valves that physically open and close to regulate fluid passage. Flexible membrane valves may operate through controlled deformation that opens or closes channel pathways. Controlled deformation may be achieved through external pressure application ranging from about 1 N to about 10 N of applied force, without limitation. Check valves may operate through one-way flow mechanisms that prevent backflow while allowing forward fluid movement. One-way flow mechanisms may utilize spring-loaded components or flexible flaps that respond to pressure differentials across the valve. Fluidic valve 736 may be any valve described herein and may allow for precise control of fluid timing and volume in first channel 712. Valve operation may enable metered fluid delivery to downstream assay components by controlling when and how much interstitial fluid flows through the serpentine geometry of first channel 712. Controlled fluid flow may result in volume regulation between 0.01 μL to about 10 μL at one or more valve 736 locations within first channel 712. Microfluidic system 127 may include additional volume control mechanisms including fixed volume channels that meter extracted interstitial fluid by filling channels of predetermined volume, with flow stopped using gas permeable membranes or hydrophobic coatings. Alternative volume control may be achieved through split channel configurations where extracted interstitial fluid is divided between a metered channel and a waste channel, separated by releasable vents, with integrated capillary valves or hydrophobic coatings stopping fluid flow in the metering channel. In some embodiments, volume control may utilize absorbent materials such as paper with controlled saturation volumes that wick the interstitial fluid and provide predetermined sample volumes for downstream analysis.

Fluidic valve 736 may be fluidically coupled to second channel 716. Second channel 716 may have one or more curved walls and a lumen extending therethrough which may allow for a passage of fluid through second channel 716. In some embodiments, second channel 716 has one or more curved portions and/or straight paths, similar to first channel 712. In some embodiments, second channel 716 and first channel 712 are geometrically symmetrical. In other embodiments, second channel 716 may differ geometrically from first channel 712. Fluidic valve 736 may be shut, which may prevent fluid from flowing between first channel 712 and second channel 716. In some embodiments, fluidic valve 736 may be opened, which may allow for a flow of fluid from first channel 712 into second channel 716. For instance, second channel 716 may be fluidically coupled to fluidic valve 736. Second channel 716 may provide a controlled fluid flow to assay system 722. For instance, second channel 716 may have curves and/or straight paths similar to first channel 712, which may regulate a flow of fluid therein. Flows of fluid delivered to assay system 722 via second channel 716 may be in the range of about, but not limited to, 0.01 μL to about 10 μL. Consistent flow rates provided to assay system 722 may allow for improved performance of assay system 722, such as but not limited to in the case of assay system 722 including one or more assay strips.

A biomolecule debonding agent may be disposed in an enzyme coating immobilized within second channel 716, or alternatively may be disposed in a blister pack integrated within the channel system. In some embodiments, second channel 716 may include a debonding agent. In some embodiments, a debonding agent may be disposed within assay system 722, such as within sensing strip 720A and/or 720B. A debonding agent may include, but is not limited to, enzymes that may debond small molecules from larger protein molecules. Small molecules may include, but are not limited to, estradiol, progesterone, testosterone, dihydrotestosterone, estrogen, cortisol, hydrocortisone, follicle stimulating hormones, melatonin, thyroid hormones (triiodothyronine (T3), thyroxine (T4)), growth hormone, insulin-like growth factor, aldosterone, dehydroepiandrosterone (DHEA), androstenedione, prolactin, oxytocin, vasopressin, parathyroid hormone, calcitonin, or other hormones. Small molecules may have sizes of less than about 900 Daltons (Da). Enzymes may include, but are not limited to, trypsin, chymotrypsin, pepsin, proteinase K, steroid sulfatase, glucuronidase, aromatase, reductase, elastase, collagenase, hyaluronidase, neuraminidase, phosphatase, esterase, lipase, amylase, cellulase, pectinase, combinations thereof, or other enzymes. An enzyme concentration may range from about 0.01 mg/mL to about 50 mg/mL, alternatively from about 0.5 mg/mL to about 20 mg/mL. In some embodiments, debonding agents may be immobilized within second channel 716 through physical adsorption, covalent attachment to functionalized channel surfaces, entrapment in hydrogels, encapsulation in microspheres, or cross-linking to polymer matrices. Physical adsorption may involve pre-treating channel surfaces to be hydrophilic through plasma treatment, corona discharge, UV/ozone treatment, chemical etching, or surface grafting and flowing enzyme solutions through second channel 716 where debonding agents are adsorbed onto channel walls. In some embodiments, the biomolecule debonding agent may be separate from the microfluidic system, such as but not limited to being part of a detection kit.

Covalent attachment may create thin coating layers on functionalized surfaces using silane chemistry, carbodiimide coupling, click chemistry, biotin-streptavidin interactions, or other coupling methods that provide improved stability and reproducibility of debonding agent immobilization. A material of second channel 716 may be glass, silica, polymer-based materials including polydimethylsiloxane (PDMS), polymethyl methacrylate (PMMA), polycarbonate, cyclic olefin copolymer (COC), polystyrene, polyethylene terephthalate (PET), or ceramic materials, with polymer-based materials being most common and requiring pre-treatment through plasma treatment, chemical modification, or other methods to create a more hydrophilic surface for enzyme immobilization. A length of immobilized surface may be between about 1 mm to about 50 mm, alternatively between about 0.5 mm to about 100 mm, or in some embodiments between about 2 mm to about 25 mm. In some embodiments, second channel 716 may include an integrated blister pack, breakable ampule, frangible seal, and/or dissolvable membrane storing reagents whose function is to debond small biomolecules from larger protein molecules. A blister pack may be pierced, ruptured, or activated through a user applying a vertical force from about 1N to about 20N, alternatively from about 1N to about 15N, and/or through thermal activation, electrical activation, magnetic activation, and/or chemical dissolution, without limitation. A volume stored in a blister pack may vary between about 1 μL to about 100 μL, alternatively between about 0.5 μL to about 500 μL, or in some embodiments between about 5 μL to about 50 μL, without limitation. A blister pack may contain debonding solutions including enzymes, chemical reagents, chaotropic agents, detergents, reducing agents, oxidizing agents, pH modifiers, ionic strength modifiers, or combinations thereof designed to disrupt biomolecule-protein bonds. A blister pack may be a frangible container with a thin membrane or seal that ruptures when sufficient force is applied, releasing the stored reagents into the fluidic channel for mixing with the extracted interstitial fluid. A blister pack may contain debonding solutions with volumes ranging from about 1 μL to about 100 μL and may require vertical activation forces ranging from about 2N to about 8N for membrane rupture.

A length of a fluidic channel containing a blister pack may range from about 1 mm to about 50 mm to provide mixing and reaction time for a biomolecule debonding processes. In some embodiments, debonding may be achieved through physical methods including sonication, mechanical agitation, temperature cycling, pressure cycling, or electromagnetic field application. In both chemical and physical embodiments, second channel 716 may include integrated filters, membranes, size exclusion columns, or molecular sieves configured to capture large protein molecules while allowing smaller biomolecule molecules to pass through. Integrated filters may include regenerated cellulose membranes, polyether sulfone membranes, polyvinylidene fluoride membranes, nylon membranes, cellulose, cellulose acetate, or other selective membranes with mesh sizes of about 5 kDa to about 10 kDa, alternatively between about 3 kDa to about 30 kDa, or in some embodiments with molecular weight cutoffs between about 1 kDa to about 50 kDa. Channel surfaces of any fluidic channel described herein may be made or coated with hydrophilic materials including but not limited to, polyethylene glycol, polyvinyl alcohol, zwitterionic polymers, or other hydrophilic coatings to facilitate capillary-driven transport, with contact angles between about but not limited to, 5 to about 25 degrees, alternatively between about 0 to about 45 degrees. Debonding agents may facilitate separation of target biomolecules from binding proteins including sex hormone-binding globulin, corticosteroid-binding globulin, thyroid-binding globulin, albumin, or other carrier proteins in interstitial fluid, blood, saliva, urine, or other biological fluids, thereby increasing availability of free hormones for detection by downstream assay components.

Still referring to FIG. 7, in use, one or more biomolecules disposed in fluid received at second channel 716 may be debonded through debonding agents, leaving free small molecules within fluid travelling through second channel 716. For instance, fluid may enter second channel 716 initially with small molecules bonded to one or more other molecules, and may become debonded from the one or more other molecules leaving only the small molecules within the fluid travelling through second channel 716. small molecules may travel through second channel 716 and into assay system 722. Assay system 722 may include any component configured to generate an indication of a biomolecule level present within fluid. For instance, in some embodiments, assay system 722 may include one or more sensing strips. Lateral flow assay strips may operate through capillary-driven flow of fluid samples across a porous membrane containing immobilized affinity binding agents at specific test zones. For instance, lateral flow assay strips may include a sample application pad where fluid is introduced, a conjugate pad containing labeled detection reagents, a nitrocellulose membrane with immobilized capture agents at test and control zones, and an absorbent pad that maintains continuous fluid flow. When target biomolecules are present in the applied sample, they may bind to labeled reagents in the conjugate pad and may be subsequently captured by immobilized affinity binding agents at the test zone, forming a detectable signal that indicates biomolecule presence and concentration. Sensing strips may be, but are not limited to, lateral flow assay (LFA) strips, electrochemical strips, fluorescent strips, molecularly imprinted polymer (MIP) strips, surface plasmon resonance (SPR) strips, or other types of sensing strips. In some embodiments, assay system 722 includes a single sensing strip. In other embodiments, assay system 722 may include two or more sensing strips, such as first sensing strip 720A and second sensing strip 720B. First sensing strip 720A may be fluidically coupled to second channel 716 through first assay channel 740A. Second sensing strip 720B may be fluidically coupled to second channel 716 through second assay channel 740B. For instance, second channel 716 may split into first assay channel 740A and second assay channel 740B. In some embodiments, debonded fluid may flow evenly from second channel 716 into each of assay channels 740A-B. In other embodiments, assay channel 740A may receive more fluid than assay channel 740B, or vice versa.

First sensing strip 720A and/or second sensing strip 720B may be configured to generate a quantitative indication of a level of one or more biomolecules in fluid received from a user. A quantitative indication may be a visual representation of an amount of one or more biomolecules present in fluid, such as but not limited to interstitial fluid. For instance, a quantitative indication may include different colors, color intensities, hues, saturation levels, or color patterns that may correspond to specific amounts and/or types of biomolecules present in fluid. In some embodiments, a single color may be representative of two or more biomolecules. For instance, a red, blue, green, or color combination thereof may represent any biomolecule described throughout this disclosure, without limitation. Different biomolecule types may be distinguished through distinct color schemes, where estradiol may generate blue or cyan colorations, progesterone may produce yellow or amber hues, testosterone may create red or magenta colors, luteinizing hormone may exhibit green or emerald tones, follicle stimulating hormone may display purple or violet shades, cortisol may manifest as orange or coral colors, and thyroid hormones may appear as pink or rose colorations. Biomolecule concentration levels may be indicated through color intensity variations, where higher biomolecule concentrations produce deeper, more saturated colors and lower concentrations generate lighter, more pastel tones. Color gradients may provide semi-quantitative measurements, with color transitions from light to dark indicating concentration ranges from low to high levels. Multi-biomolecule detection may utilize color mixing or pattern combinations, where simultaneous presence of multiple biomolecules creates composite colors or spatially separated color zones on sensing strips. Color hue shifts may indicate concentration changes over time, with gradual color transitions from one hue to another representing dynamic biomolecule level fluctuations. Amounts of biomolecules in a fluid may range from about, but not limited to, 0.1 pmol/L to about 1000 nmol/L in molar units, or equivalently from about 1 ng/dL to about 1,000 ng/dL or greater in mass-based units.

First sensing strip 720A may be a lateral flow assay utilizing a porous strip in which affinity binding agents are disposed, e.g., immobilized onto defined regions of the membrane. For instance, first sensing strip 720A may generate a color coded pattern, fluorescent signal, electrochemical response, luminescent output, or other detectable signal indicative of a presence, absence, or concentration of one or more biomolecules. Second sensing strip 720B may be configured to generate an indication of a level of one or more biomolecules in fluid received from a user. In some embodiments, first sensing strip 720A may be configured for detection of low concentration ranges of biomolecules, such as concentrations in ranges of, but not limited to, about 0.1 pmol/L to about 10 pmol/L, while second sensing strip 720B may be configured for detection of high concentration ranges of biomolecules, such as concentrations in ranges of, but not limited to, about 10 pmol/L to about 1000 nmol/L. This dual-range configuration enables accurate quantification across the full physiological range of biomolecule concentrations that may be encountered in interstitial fluid monitoring applications. For instance, sensing strip 720B may generate a color coded pattern, quantitative readout, semi-quantitative indication, or qualitative result indicative of a presence, absence, or concentration of one or more biomolecules. In some embodiments, first sensing strip 720A and second sensing strip 720B are the same type of sensing strip for redundancy and quality control. In other embodiments, first sensing strip 720A may differ in sensing strip type, target biomolecule, detection mechanism, or sensitivity range than second sensing strip 720B. In some embodiments, first sensing strip 720A may be configured to generate an indication of a presence or absence of a first set of biomolecules, which may include hormones such as, but not limited to, estradiol, progesterone, testosterone, luteinizing hormone, or follicle stimulating hormone and second sensing strip 720B may be configured to generate an indication of a presence or absence of a second set of biomolecules different than the first set of biomolecules which may include hormones such as, but not limited to, cortisol, thyroid hormones, growth hormone, insulin, or other metabolic biomolecules.

In other embodiments, second sensing strip 720B may be redundant to first sensing strip 720A, which may provide assurance, validation, or confirmation in detection of a presence or absence of one or more biomolecules within fluid of a user. Detection of biomolecules may occur through competitive assay formats, sandwich assay formats, inhibition assay formats, or other immunoassay configurations, or displacement assay formats. In a competitive format, the test zone contains immobilized biomolecule, biomolecule analog, or biomolecule conjugate, and when target biomolecules are present in the fluid, they compete for binding with labeled affinity binding agents including antibodies, aptamers, or other recognition elements, resulting in a weaker signal that inversely correlates to biomolecule concentration. In a sandwich format, the test zone contains capture agents including antibodies, aptamers, molecularly imprinted polymers, or other selective binding agents that specifically bind target biomolecules, and when biomolecules are present, they bind to labeled reagents in the conjugate pad and are subsequently captured at the test zone by immobilized capture agents, forming a detectable complex that directly correlates to biomolecule concentration. In a displacement format, labeled analogs are displaced from binding sites by target biomolecules, creating a signal proportional to biomolecule concentration. The affinity binding agents may include monoclonal antibodies, polyclonal antibodies, antibody fragments (Fab, F(ab′)2, scFv), aptamers, molecularly imprinted polymers, affibodies, nanobodies, peptides, proteins, or other selective recognition elements immobilized onto defined regions of the sensing strip membrane through passive adsorption, covalent attachment, biotin-streptavidin coupling, or other immobilization methods. Signal generation may utilize various labels including gold nanoparticles, silver nanoparticles, latex beads, magnetic beads, quantum dots, upconversion nanoparticles, fluorescent dyes, enzymes (horseradish peroxidase, alkaline phosphatase), chemiluminescent reporters, electroactive species, or other detectable markers. The sensing strips may include imaging readout systems including portable handheld readers, integrated mobile devices with cameras, spectrophotometers, fluorometers, electrochemical analyzers, or other detection instruments with processing software, algorithms, or artificial intelligence to provide quantitative analysis by converting measured signal intensity, color changes, electrical signals, or other measurable parameters into numerical values, concentration ranges, or categorical results that correlate to biomolecule concentration levels.

The readout systems may include calibration curves, reference standards, internal controls, or machine learning algorithms to improve accuracy and precision of biomolecule quantification. The lateral flow assay strips may comprise multiple functional layers including a sample pad for receiving and treatment of the applied sample, a conjugate pad for storing labeled affinity reagents, a nitrocellulose membrane containing immobilized test and control zones, and an absorbent pad that maintains capillary-driven flow. Each component may be pre-treated with surfactants, blocking molecules, or stabilizers to optimize flow characteristics, minimize non-specific binding, and enhance sensitivity. The strip components may have varying lengths and materials optimized for specific biomolecule detection requirements, with sample pads, conjugate pads, and absorbent pads dimensioned to provide appropriate fluid handling capacity and flow rates for the target biomolecules.

With continued reference to FIG. 7, microfluidic system 128 may include pressure membrane 732 fluidically coupled to output channel 728. Output channel 728 may be fluidically coupled to first assay output channel 724A and/or second assay output channel 724B. First assay output channel 724A and second assay output channel 724B may output similar or the same type of fluids. For instance, fluids may include, but are not limited to, processed sample fluids, reagent solutions, buffer solutions, wash solutions, waste fluids, purified biomolecules, concentrated samples, diluted samples, and/or reaction products. In some embodiments, first sensing strip 720A may operate in parallel with second sensing strip 720B, which may cause identical or near identical fluid compositions to exit through first assay output channel 724 and second assay output channel 724B. In other embodiments, first sensing strip 720A may operate in series and/or sequentially with second sensing strip 720B, which may cause different fluid compositions to enter first assay output channel 724A compared to second assay output channel 724B. Flow rates through first assay output channel 724A and/or second assay output channel 724B may range from approximately 0.1 μL/min to 1000 μL/min.

Pressure membrane 732 may stretch, which may cause negative pressure to be applied to microfluidic system 128. Negative pressure may cause a flow of fluid from fluidic channels 708 into assay system 722 due to a pressure difference between fluidic channels 708 and assay system 722. A user may push on a pressure actuator repeatedly, which may cause rapid increase in negative pressure within microfluidic system 128, allowing for increased flow of fluid to occur throughout channels of microfluidic system 128.

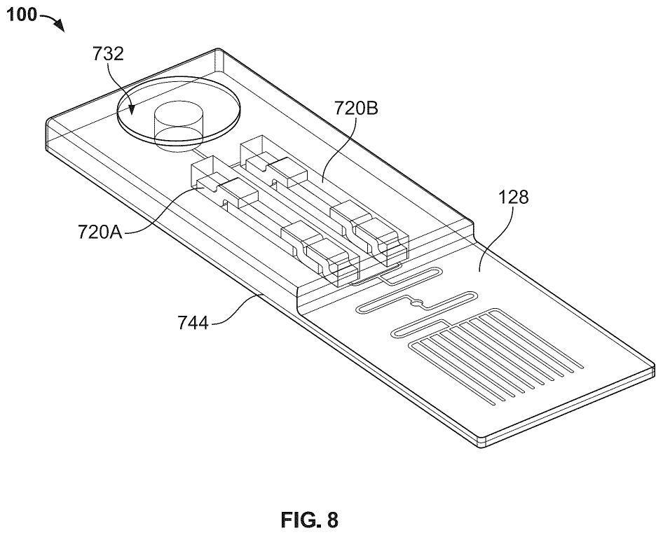

Referring now to FIG. 8, a top side perspective view of device 100 is presented. Device 100 may include fluidic channels 708, assay system 722, first sensing strip 720A, second sensing strip 720B, and pressure membrane 732 as described above with reference to FIG. 7.

In some embodiments, housing 744 (also referred to as “cartridge 744”) may be transparent or semi-transparent, which may allow for visual observation of internal components and processes within microfluidic system 128. In some embodiments, transparent housing 744 may be configured to slide into a detection device, such as through a port or slot of the detection device, enabling automated positioning and analysis of the assay system components. In some embodiments, transparent housing 744 may be removable from through sliding mechanisms, snap-fit connections, threaded interfaces, or other mechanical coupling methods that allow selective access to internal components while maintaining environmental protection when secured. Housing 744 may be fabricated from transparent materials such as, but not limited to, clear polycarbonate, acrylic (polymethyl methacrylate (PMMA)), clear polystyrene, transparent polyethylene terephthalate (PET), clear cyclic olefin copolymer (COC), or other optically clear polymers. In some embodiments, housing 744 may have a light transmittance of about 85% to about 95% or greater in the visible light spectrum ranging from about 400 nm to about 700 nm. Transparent housing 744 may enable direct visual inspection of fluid flow through fluidic channels 708, monitoring of assay system 722 operation, and observation of color changes or other visual indications generated by first sensing strip 720A and second sensing strip 720B without requiring removal of housing 744 or disassembly of device 100. In some embodiments, housing 744 may include removable sections, access ports, or detachable components that enable removal of assay system components for external analysis while maintaining the integrity of the microfluidic system.

In some embodiments, housing 744 may include removable sections, access ports, or detachable components that enable removal of assay system components for external analysis while maintaining the integrity of microfluidic system 128. Removable sections may include hinged covers, sliding panels, snap-fit closures, or threaded caps that provide selective access to specific regions of housing 744 where assay system 722 is positioned. Access ports may be circular, rectangular, or other shaped openings with diameters or dimensions ranging from about 5 mm to about 30 mm, sized to accommodate removal of sensing strips 720A-B or other assay components without compromising the sealed environment of microfluidic system 128. Detachable components may include removable top covers, side panels, or bottom sections that can be separated from housing 744 through mechanical fasteners, adhesive bonds, or interference fits. The removable sections, access ports, or detachable components may incorporate sealing mechanisms such as gaskets, O-rings, or compression seals that maintain environmental protection for microfluidic system 128 when closed while allowing convenient access when opened. In some embodiments, the access ports may include hinged doors, sliding covers, or removable plugs that can be opened to extract sensing strips 720A-B for external imaging analysis using smartphone cameras, handheld readers, or other detection devices. The port design may ensure that removal of assay system components does not disrupt ongoing fluid flow processes or compromise sterility of remaining microfluidic channels 708 and associated components within housing 744. In some embodiments, individual sensing strips 720A-B may be separately removed through the access ports for independent analysis, while in other embodiments, the entire assay system 722 cartridge containing both sensing strips may be removed as a single unit for external imaging analysis.

In some embodiments, transparent housing 744 may facilitate real-time monitoring of biomolecule detection processes occurring within assay system 722. For instance, first sensing strip 720A and second sensing strip 720B may generate visual indications including color changes, line formations, intensity variations, or other optical signals that may be directly observable through transparent housing 744. Visual indications may include, but are not limited to, colorimetric changes ranging from colorless to colored states, formation of colored lines or bands at test zones, development of control lines for assay validation, intensity gradations corresponding to biomolecule concentration levels, or fluorescent signals under appropriate illumination conditions. In competitive assay formats, visual indications may manifest as inverse relationships between biomolecule concentration and signal intensity, where higher biomolecule concentrations result in weaker visual signals at test zones of sensing strips 720A-B. In sandwich assay formats, visual indications may manifest as direct relationships between biomolecule concentration and signal intensity, where higher biomolecule concentrations result in stronger visual signals at test zones. Color changes may occur within time periods ranging from about 30 seconds to about 30 minutes after fluid contact with sensing strips 720A-B, alternatively between about 1 minute to about 15 minutes, or in some embodiments between about 2 minutes to about 10 minutes. The transparent nature of housing 744 may eliminate the need for housing removal or disassembly to observe assay results, thereby maintaining sterility, preventing contamination, and preserving the integrity of microfluidic system 128 during biomolecule detection processes.

Transparent housing 744 may incorporate optical features designed to enhance visualization of sensing strips 720A-B and assay system 722. For instance, housing 744 may include magnification zones, optical lenses, light-focusing elements, anti-reflective coatings, or light-diffusing surfaces to improve visibility of visual indications generated by sensing strips 720A-B. Magnification zones may provide magnification factors of about 1.5× to about 5× or greater, allowing for enhanced observation of subtle color changes, line formations, or intensity variations. In some embodiments, housing 744 may include integrated lighting elements such as light-emitting diodes (LEDs), fiber optic illumination, or reflective surfaces to provide consistent illumination of sensing strips 720A-B. Integrated lighting may emit light in wavelength ranges optimized for specific detection modalities, including white light for colorimetric detection, and/or ultraviolet light for fluorescent detection. Light sources may operate at intensities ranging from about 10 lux to about 10,000 lux, alternatively between about 500 lux to about 5,000 lux, to provide adequate illumination without causing photobleaching or thermal damage to sensing strips 720A-B. Housing 744 may include viewing windows, observation ports, or transparent sections strategically positioned to align with test zones and control zones of sensing strips 720A-B, ensuring optimal visualization of biomolecule detection results. Viewing windows may have dimensions ranging from about 5 mm×5 mm to about 20 mm×20 mm, alternatively between about 3 mm×3 mm to about 30 mm×30 mm, sized to encompass the active detection areas of sensing strips 720A-B while maintaining structural integrity of housing 744.

In some embodiments, transparent housing 744 may enable monitoring of fluid flow dynamics throughout microfluidic system 128. Visual observation through transparent housing 744 may allow for real-time assessment of fluid movement through fluidic channels 708, detection of flow blockages or irregularities, monitoring of fluid accumulation in channel curves or junctions, and verification of proper fluid delivery to sensing strips 720A-B. Fluid flow visualization may be enhanced through the use of colored fluids, fluorescent tracers, or contrast agents that improve visibility of fluid movement through transparent channels. In some embodiments, fluidic channels 708 may be fabricated from transparent or translucent materials including clear polydimethylsiloxane (PDMS), glass, clear polycarbonate, or other optically clear materials that complement the transparency of housing 744. Transparent fluidic channels 708 may have wall thicknesses ranging from about 0.1 mm to about 2 mm, alternatively between about 0.05 mm to about 5 mm, optimized to provide structural integrity while maintaining optical clarity. The combination of transparent housing 744 and transparent fluidic channels 708 may enable comprehensive visual monitoring of the entire fluid handling process from microneedle extraction through final biomolecule detection at sensing strips 720A-B. Visual monitoring may facilitate troubleshooting of device operation, quality control during manufacturing, and user confidence in proper device function during biomolecule monitoring applications.

Transparent housing 744 may facilitate integration with external detection systems and imaging devices. For instance, transparent housing 744 may be compatible with smartphone cameras, digital microscopes, handheld scanners, or other imaging devices that may capture images of sensing strips 720A-B through housing 744 without requiring device disassembly. External imaging devices may utilize various detection modalities including visible light photography, fluorescence imaging, chemiluminescence detection, or spectroscopic analysis to quantify biomolecule levels based on visual indications from sensing strips 720A-B. In some embodiments, transparent housing 744 may include alignment features, positioning guides, or reference markers to ensure consistent positioning of external imaging devices relative to sensing strips 720A-B. Alignment features may include printed fiducial markers, embossed reference points, or geometric features that facilitate automated image analysis and biomolecule quantification. Transparent housing 744 may be designed to minimize optical distortions, reflections, or aberrations that could interfere with accurate imaging of sensing strips 720A-B. Housing 744 may incorporate anti-glare coatings, polarizing filters, or surface textures to reduce unwanted reflections and improve image quality. The optical properties of transparent housing 744 may be optimized for specific wavelengths of light used in biomolecule detection, including visible light wavelengths for colorimetric assays, ultraviolet wavelengths for fluorescent assays, or infrared wavelengths for specialized detection modalities.

A transparent nature of housing 744 may provide additional functional benefits beyond visual observation of sensing strips 720A-B. For instance, transparent housing 744 may enable monitoring of pressure membrane 732 operation, allowing users to visually confirm proper membrane stretching and pressure generation during device operation. Visual confirmation of pressure membrane 732 function may provide user feedback regarding successful activation of negative pressure systems and proper fluid flow initiation. In some embodiments, transparent housing 744 may include graduated markings, measurement scales, or volume indicators that allow for visual estimation of fluid volumes within microfluidic system 128. Volume indicators may be calibrated to correspond to specific fluid volumes ranging from about 0.1 μL to about 100 μL, enabling users to monitor fluid collection progress and verify adequate sample volumes for biomolecule detection. Transparent housing 744 may facilitate quality control during device manufacturing by enabling visual inspection of internal component alignment, channel integrity, and assembly quality without destructive testing. Manufacturing quality control may include verification of proper sensing strip 720A-B positioning, confirmation of fluidic channel 708 continuity, and assessment of overall device assembly quality. In some embodiments, transparent housing 744 may be selectively opaque in certain regions to protect light-sensitive components while maintaining transparency in critical observation areas around sensing strips 720A-B and key fluidic pathways. Transparent housing 744 may be configured to interface with a camera box system for automated analysis of sensing strips 720A-B. The camera box may detect insertion of device 100 through physical sensors, optical sensors, or laser sensors that recognize when the device is properly positioned for imaging analysis. In some embodiments, device 100 may include a unique quality control (QC) code that enables identification and calibration by the camera box imaging system. The QC code may be applied to device casing 144 or transparent housing 744 through pad printing, laser etching, inkjet printing, or adhesive stickers, and may contain device-specific information including manufacturing date, lot number, calibration parameters, or assay configuration data. The camera box may include an internal reference color chart for system calibration prior to imaging analysis, ensuring consistent and accurate measurement of visual indications from sensing strips 720A-B. The imaging system may capture signal intensity measurements from both control lines and test lines of lateral flow assays within assay system 722, enabling quantitative calculation of biomolecule concentrations based on comparative analysis of line intensities. Transparent housing 744 may be optimized to allow transmission of illumination light required for imaging while minimizing reflection, optical distortion, and haze that could reduce image quality during automated analysis. The camera box system may utilize various imaging modalities including visible light photography, fluorescence imaging, or spectroscopic analysis to quantify biomolecule levels based on visual indications captured through transparent housing 744.

In some embodiments, device 100 may be provided as part of a biomolecule detection kit that includes additional components for biomolecule level analysis. The kit may include a detection system configured to detect biomolecule levels based on indications generated by sensing strips 720A-B within assay system 722. The detection system may include various imaging devices including smartphone cameras, handheld readers, digital microscopes, or other optical detection instruments capable of capturing and analyzing visual indications from the sensing strips. The detection system may be configured to perform colorimetric analysis by capturing images of color changes, line formations, or intensity variations generated by the sensing strips and converting these visual signals into quantitative biomolecule concentration measurements. In some embodiments, the detection system may include specialized software applications, algorithms, or artificial intelligence components that process captured images to determine biomolecule levels with improved accuracy and precision. The detection system may also be configured to detect fluorescence, chemiluminescence, or other non-colorimetric signals from the sensing strips, requiring appropriate excitation sources and detectors for such detection modalities. The kit configuration enables users to perform complete biomolecule monitoring workflows by combining the microfluidic device 100 with external detection capabilities, providing a comprehensive solution for intermittent biomolecule monitoring applications.

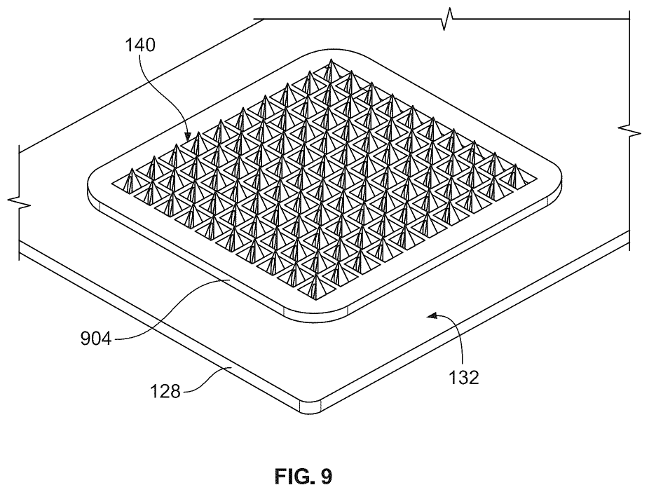

Referring now to FIG. 9, an embodiment of a microneedle assembly 132 may include microneedles 104 described above with reference to FIG. 3. Microneedle assembly 132 may include rib 904. Rib 904 may be positioned around a perimeter of microneedle array 132 and may be designed to create positive pressure on a skin surface of a user during device operation. Rib 904 may be a raised structural element that extends beyond a bottom surface of microneedle assembly 132, creating a contact interface between device 100 and the user's skin. In some embodiments, rib 904 may have a height ranging from about 0.5 mm to about 5 mm, alternatively between about 0.1 mm to about 10 mm, measured from the bottom surface of microneedle assembly 132. The height of rib 904 may be optimized to provide adequate skin contact pressure while allowing microneedles 140 to achieve proper penetration depths of about 500 μm to about 1500 μm into the skin surface. Rib 904 may be fabricated from the same material as microneedle assembly 132 or may include different materials such as rigid plastics, elastomers, or composite materials that provide enhanced structural support and pressure distribution. The cross-sectional shape of rib 904 may be rectangular, circular, oval, or other geometric configurations that optimize skin contact area and pressure distribution characteristics. Rib 904 may function to create positive pressure on a skin surface ranging from about 10 kPa to about 90 kPa, with a nominal operating pressure of about 50 kPa. Rib 904 may create a pressure gradient that facilitates fluid flow into microneedles by establishing pressure differentials between the skin surface and the microneedle lumens, enhancing interstitial fluid extraction efficiency. A pressure gradient created by rib 904 may range from about, but not limited to, 1 kPa to about 50 kPa. A pressure gradient may be between microneedle array 132 and a portion of a user. A pressure gradient may facilitate flow of fluid into one or more microneedles of microneedle array 132 via differences in pressure between microneedle array 132 and bodily pressure where microneedle array 132 may be applied.

In some embodiments, microneedle array 132 may function to mediate skin stretch by creating controlled tension that maintains skin mechanical properties including but not limited to elasticity, viscoelasticity, and tensile strength of skin. For instance, skin that may contact microneedle array 132 may be elastic, which may reduce penetration effectiveness of one or more microneedles. Microneedle array 132 may keep a patch of skin stretched which may facilitate penetration of one or more microneedles of microneedle array 132. For instance, microneedle array 132 may impose localized tensile strain on a portion of skin surrounding one or more needle insertion sites. Localized tensile strain may be between about, but not limited to, 1% to about 50% or greater of a baseline tensile strength of skin. Localized tensile strain may counteract skin's intrinsic deformability, anisotropy, and/or time-dependent viscoelastic response, which may otherwise cause surface displacement and/or resistance to consistent needle penetration. In some embodiments, a spacing between microneedles of microneedle array 132 may be between about, but not limited to, 500 μm to about 5,000 μm or greater than about 5,000 μm. A spacing between two or more microneedles of microneedle array 132 may act to mediate a stretching of skin that microneedle array 132 may contact. In some embodiments, a spacing between two or more microneedles of microneedle array 132 may allow for uniform fluidic resistance across microneedle array 132. Uniform fluidic resistance may ensure a uniform flow of fluid into one or more microneedles of microneedle array 132. Fluidic resistances may be between about, but not limited to, 1 Pa·s/m³ to about 100 Pa·s/m³ or greater. In some embodiments, fluidic resistance of each microneedle of microneedle array 132 may be about the same, such as within a 0% to 10% difference. In other embodiments, a fluidic resistance across two or more microneedles of microneedle array 132 may differ.

Rib 904 may function to create positive pressure on the skin surface ranging from about 10 kPa to about 90 kPa, with a nominal operating pressure of about 50 kPa, as described in the disclosure. The positive pressure may be generated through mechanical coupling with pressure actuator 112, where downward force applied by a user causes rib 904 to compress against the skin surface. In some embodiments, rib 904 may be mechanically coupled to spring 160 or similar elastic elements that provide controlled compression force when pressure actuator 112 is depressed. The compression mechanism may utilize a weighted mass, compression spring, or pneumatic system that applies consistent pressure to rib 904, thereby creating uniform skin contact pressure around the microneedle insertion area. Rib 904 may serve a dual function by stretching the skin surface to create tension when microneedles 140 are deployed, which may improve puncturing performance and reduce the force required for skin penetration. The tensioned skin surface created by rib 904 may provide a more stable platform for microneedle insertion, reducing lateral movement and ensuring consistent penetration depths across microneedle array 132. Additionally, rib 904 may create a sealed or semi-sealed environment around microneedle array 132 that enhances interstitial fluid extraction by preventing fluid loss and maintaining localized pressure differentials that drive fluid flow into the microneedles. During device application, rib 904 and the surrounding device structure may impose localized tensile strain on the skin surface surrounding microneedle insertion sites, typically ranging from about 5% to about 20% strain. This pre-stretching may be necessary to counteract skin's intrinsic deformability, anisotropy, and time-dependent viscoelastic response, which may otherwise cause surface displacement and resistance to consistent microneedle penetration. The induced strain may result from a combination of design factors including: (1) dynamics of device application, including rate of deployment through user activation of needle inserter 116; (2) placement and distribution of adhesive patch 108, which may anchor and tension tissue; and (3) geometric configuration of microneedle array 132 and rib 904, which may guide how pressure is transferred to the skin surface during device operation.

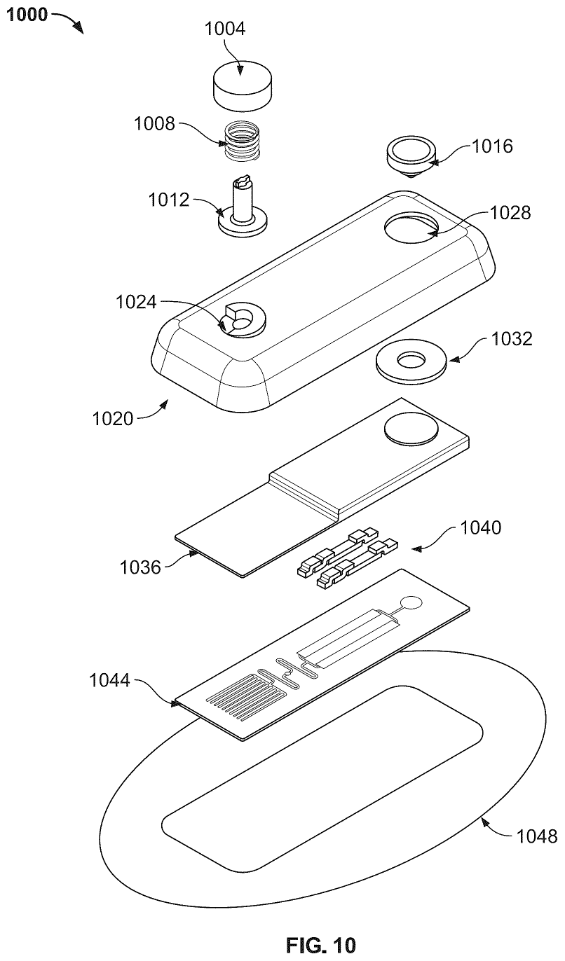

Referring to FIG. 10, an exploded view of a device 1000 for intermittent biomolecule monitoring is presented. Device 1000 may include needle inserter 1004, spring 1008, plunger 1012, and pressure actuator 1016, each of which may be assembled into device casing 1020. Device 1000 may include overmold 1032, which may be positioned between device casing 1020 and microfluidic cap 1036. In some embodiments, overmold 1032 may connect to pressure actuator 1016 and/or casing 1020. Assay system 1040 may be positioned between microfluidic cap 1036 and microfluidic system 1044. One or more components of device 1000 may be coupled to adhesive patch 1048.

Needle inserter 1004 may be positioned at a proximal end of device 1000 and may be configured to receive kinetic energy from a user to initiate deployment of microneedle array 132. Needle inserter 1004 may be circular or other shaped element with a diameter ranging from about 5 mm to about 20 mm, alternatively between about 3 mm to about 30 mm. In some embodiments, needle inserter 1004 may be fabricated from rigid materials such as polycarbonate, acrylonitrile butadiene styrene (ABS), polyoxymethylene (POM), or other durable polymers that can withstand repeated user activation forces. Needle inserter 1004 may be designed to travel vertically by about 1 mm to about 10 mm when depressed by a user, with forces required for activation ranging from about 1 N to about 50 N. The surface of needle inserter 1004 may include textured regions, ridges, or other tactile features to enhance grip and provide user feedback during activation. In some embodiments, needle inserter 1004 may include visual indicators such as colored regions, symbols, or text that guide proper user interaction and confirm successful activation of the microneedle deployment mechanism.

Spring 1008 may be mechanically coupled to needle inserter 1004 and may provide controlled resistance against downward movement during user activation. Spring 1008 may be a compression spring, helical compression spring, conical spring, or wave spring fabricated from materials such as stainless steel, titanium, or other spring materials that maintain elastic properties over repeated use cycles. In some embodiments, spring 1008 may have a spring constant ranging from about 1 N/mm to about 50 N/mm, optimized to prevent accidental deployment while allowing intentional user activation. The spring constant may be selected to provide tactile feedback to the user, indicating successful compression and subsequent release for microneedle deployment. Spring 1008 may be wrapped around a base portion of needle inserter 1004 in a clockwise or counter-clockwise direction, with coil dimensions optimized for the available space within device casing 1020. When compressed by user force applied to needle inserter 1004, spring 1008 may store potential energy that is subsequently released to drive plunger 1012 and deploy microneedle array 132 with controlled force and velocity.

Plunger 1012 may be mechanically coupled to spring 1008 and microneedle array 132, serving as an intermediate component that transfers force from spring 1008 to the microneedle deployment mechanism. Plunger 1012 may be round, circular, cylindrical, or other geometric shapes with dimensions optimized to fit within device casing 1020 while providing adequate surface area for force transmission. In some embodiments, plunger 1012 may be fabricated from materials such as rubber, stainless steel, aluminum, or polymer-based materials including polyoxymethylene (POM) or polyetheretherketone (PEEK) that provide durability and biocompatibility. Plunger 1012 may have a length ranging from about 5 mm to about 25 mm and a diameter ranging from about 3 mm to about 15 mm, sized to provide mechanical stability during force transmission while minimizing overall device volume. When spring 1008 decompresses following user activation of needle inserter 1004, plunger 1012 may receive downward force and translate this force to microneedle array 132, causing deployment and insertion of individual microneedles into the user's skin to depths of about 500 μm to about 1500 μm for interstitial fluid access.

Pressure actuator 1016 may be positioned at a proximal end of device 1000, potentially adjacent to or integrated with needle inserter 1004, and may be configured to control pressure generation within microfluidic system 1044. Pressure actuator 1016 may be a circular, rectangular, or other shaped button that receives kinetic force from a user in the form of pressing motions directed toward a bottom surface of device casing 1020. In some embodiments, pressure actuator 1016 may travel vertically by about 1 mm to about 200 mm or greater when activated, with forces required for operation ranging from about 2 N to about 8 N. Pressure actuator 1016 may be mechanically coupled to pressure membrane components within microfluidic system 1044, allowing user activation to create negative pressure ranging from about −1 kPa to about −5 kPa, alternatively up to about −50 kPa when integrated with valve systems. The negative pressure generated by pressure actuator 1016 may facilitate fluid flow from microneedle array 132 through microfluidic channels by creating pressure differentials that drive interstitial fluid movement toward assay system 1040. Pressure actuator 1016 may include tactile feedback mechanisms, visual indicators, or audible signals that confirm successful activation and pressure generation within the microfluidic system.

Device casing 1020 may provide structural housing and protection for all internal components of device 1000, including needle inserter 1004, spring 1008, plunger 1012, pressure actuator 1016, and downstream microfluidic components. Device casing 1020 may be fabricated from rigid materials such as polycarbonate, acrylonitrile butadiene styrene (ABS), polyoxymethylene (POM), polyetheretherketone (PEEK), or other durable polymers that provide mechanical protection while maintaining biocompatibility. In some embodiments, device casing 1020 may have dimensions ranging from about 25.4 mm to about 127 mm in length and width, with height ranging from about 2.54 mm to about 76.2 mm, optimized to accommodate internal components while maintaining portability and user comfort. Device casing 1020 may include internal compartments, mounting features, or alignment guides that ensure proper positioning and mechanical coupling of needle inserter 1004, spring 1008, plunger 1012, and pressure actuator 1016 during assembly and operation. The casing may incorporate sealing features, gaskets, or barriers that protect internal components from environmental conditions including air, dust, water, humidity, and light while maintaining sterility of the microfluidic system. In some embodiments, device casing 1020 may be transparent or semi-transparent to enable visual observation of internal component operation and fluid flow processes.

Overmold 1032 may be positioned between device casing 1020 and microfluidic cap 1036, serving as an interface component that provides sealing, alignment, and mechanical coupling between the upper device assembly and the microfluidic system components. Overmold 1032 may be fabricated from flexible elastomeric materials such as silicone, polyurethane, or thermoplastic elastomers that provide sealing capabilities while accommodating mechanical movement of pressure actuator 1016 and associated pressure generation mechanisms. In some embodiments, overmold 1032 may create a sealed enclosure around portions of microfluidic system 1044, preventing contamination and maintaining sterility of fluid pathways. Overmold 1032 may include integrated sealing features such as O-rings, gaskets, or compression seals that interface with device casing 1020 and microfluidic cap 1036 to create hermetic seals around critical components. The overmold may include pressure membrane features that work in conjunction with pressure actuator 1016 to generate negative pressure within microfluidic channels, with membrane areas and stroke lengths optimized to achieve pressure ranges from about −1 kPa to about −5 kPa. Overmold 1032 may also include alignment features, positioning guides, or mechanical interfaces that ensure proper assembly and operation of the integrated device components.

Microfluidic cap 1036 may be positioned between overmold 1032 and assay system 1040, serving as a protective cover and interface component for the microfluidic system components. Microfluidic cap 1036 may be fabricated from rigid materials such as polycarbonate, polymethyl methacrylate (PMMA), or other transparent polymers that provide structural protection while enabling visual observation of microfluidic processes. In some embodiments, microfluidic cap 1036 may include integrated fluidic connectors, channel interfaces, or sealing features that facilitate proper coupling between pressure generation mechanisms in overmold 1032 and fluid handling components in microfluidic system 1044. The cap may incorporate viewing windows, observation ports, or transparent sections strategically positioned to enable monitoring of fluid flow, pressure membrane operation, and assay system function. Microfluidic cap 1036 may include mounting features, alignment guides, or mechanical interfaces that ensure proper positioning relative to assay system 1040 and microfluidic system 1044 during assembly and operation. In some embodiments, the cap may include integrated optical features such as magnification zones, light-focusing elements, or anti-reflective coatings that enhance visualization of downstream assay components and biomolecule detection processes.

Assay system 1040 may be positioned between microfluidic cap 1036 and microfluidic system 1044, including sensing strips and detection components configured to generate indications of biomolecule levels present in extracted interstitial fluid. Assay system 1040 may include lateral flow assay strips, electrochemical strips, fluorescent strips, or other sensing technologies that utilize affinity binding agents such as antibodies, aptamers, or other selective recognition elements to detect target biomolecules. In some embodiments, assay system 1040 may include multiple sensing strips operating in parallel or series to provide redundancy, quality control, or detection of multiple biomolecule types simultaneously. The sensing strips may be configured to generate visual indications including color changes, line formations, intensity variations, or other optical signals that correlate to biomolecule concentration levels in the processed interstitial fluid. Assay system 1040 may be designed to interface with external detection systems including smartphone cameras, handheld readers, or other imaging devices that capture and analyze visual indications for quantitative biomolecule measurement. The assay system may include control zones, reference standards, or calibration features that ensure proper assay function and enable accurate biomolecule quantification across a range of physiological concentrations.

Microfluidic system 1044 may include the core fluid handling components of device 1000, including microneedle array interfaces, fluidic channels, pressure control mechanisms, volume control systems, and debonding agent integration. Microfluidic system 1044 may be fabricated from materials such as polydimethylsiloxane (PDMS), glass, polycarbonate, polymethyl methacrylate (PMMA), or other biocompatible materials that provide chemical resistance and optical clarity for fluid handling applications. The system may include serpentine channel configurations with curved and straight path segments that provide controlled fluidic resistance, metering capabilities, and enhanced mixing of interstitial fluid with debonding agents. Microfluidic system 1044 may include integrated filters with molecular weight cutoffs ranging from about 5 kDa to about 10 kDa to separate small biomolecules from larger protein molecules following debonding processes. The system may include capillary valves, hydrophobic valves, or mechanical valves that control fluid timing and volume delivery to assay system 1040, with valve operation enabling metered fluid delivery ranging from about 0.1 μL to about 10 μL. In some embodiments, microfluidic system 1044 may include flexible membrane valves connected to controlled valve systems that open and close to enable increasing negative pressure with each stroke, achieving negative pressures ranging from about −10 kPa to about −50 kPa. The flexible membrane may be stretched by spring mechanisms upon release, with critical dimensions including membrane area, stroke length of compression, and total negative pressure generated being optimized for specific fluid handling requirements. Microfluidic system 1044 may interface with adhesive patch 1048 to provide secure attachment to the user while maintaining proper alignment of microneedle array components for effective interstitial fluid extraction and biomolecule monitoring.

Adhesive patch 1048 may provide the interface between device 1000 and the user's skin, ensuring secure attachment during biomolecule monitoring while accommodating microneedle deployment and interstitial fluid extraction. Adhesive patch 1048 may be circular, oval, rectangular, or other shapes with dimensions ranging from about 50 mm to about 300 mm in length and width, providing adequate surface area for stable device attachment. The patch may be fabricated from biocompatible materials including medical-grade adhesives such as acrylic adhesives, silicone adhesives, hydrocolloid adhesives, or polyurethane adhesives that provide secure skin attachment while minimizing irritation and enabling comfortable removal. Adhesive patch 1048 may include an aperture sized to accommodate the bottom surface of device casing 1020 or specifically sized for microneedle array deployment, allowing direct skin contact while maintaining device stability. In some embodiments, the patch may include multiple adhesive zones with different adhesive strengths, including stronger adhesives for device attachment ranging from about 0.2 N/mm to about 2.0 N/mm and gentler adhesives for skin contact ranging from about 0.08 N/mm to about 1.0 N/mm, without limitation. Adhesive patch 1048 may include removal features such as tabs, pull strips, or release liners that facilitate easy and comfortable device removal following biomolecule monitoring completion.

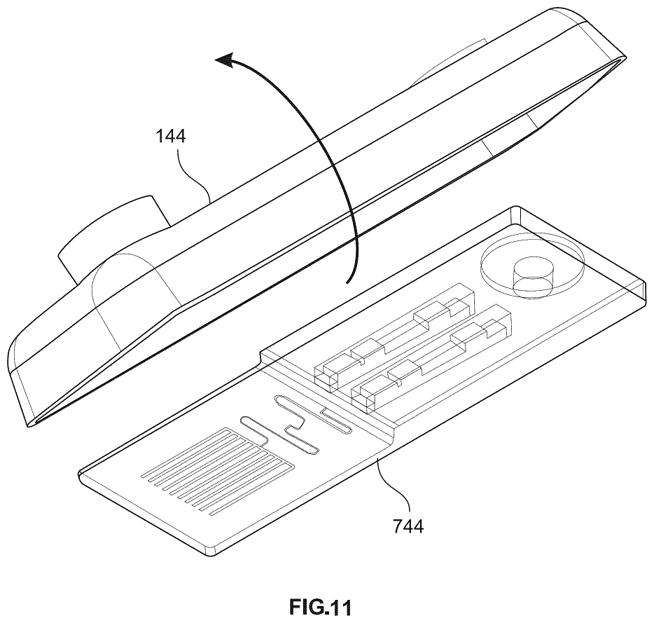

Referring now to FIG. 11, an embodiment of a removal of a device casing is presented. For instance, device casing 144 may be removable from housing 744. For instance, device casing 144 may be removably coupled to housing 744 through various mechanical coupling mechanisms that enable selective separation of the components for access to internal assay system components. Device casing 144 may be removed from housing 744 through sliding mechanisms where device casing 144 slides linearly relative to housing 744 along predetermined tracks, guides, or channels. Sliding removal may require forces ranging from about 2 N to about 15 N applied in directions parallel to the sliding axis. In alternative embodiments, device casing 144 may be coupled to housing 744 through latch mechanisms including spring-loaded latches, cam latches, or lever latches that secure the components together and release when actuated by user force. Latch mechanisms may require activation forces ranging from about 1 N to about 10 N applied to release tabs, buttons, or levers. Device casing 144 may also be removably coupled through hook mechanisms including flexible hooks, rigid hooks, or snap-fit hooks that engage with corresponding receptacles, grooves, or undercuts in housing 744. Hook release may be achieved through deflection of flexible elements or disengagement of mechanical interfaces requiring forces from about 3 N to about 12 N. In some embodiments, device casing 144 may be removed through rotational mechanisms including threaded connections, bayonet connections, or twist-lock mechanisms where device casing 144 rotates relative to housing 744 through angles ranging from about 15 degrees to about 180 degrees to achieve coupling or decoupling. Rotational removal may include torques ranging from about 0.1 N·m to about 2 N·m applied about the rotational axis. Additional removal mechanisms may include magnetic coupling where magnetic forces secure device casing 144 to housing 744 and can be overcome by applied separation forces, compression mechanisms where device casing 144 is compressed or decompressed relative to housing 744 for removal, or combination mechanisms that utilize multiple coupling methods simultaneously for enhanced security and controlled release.

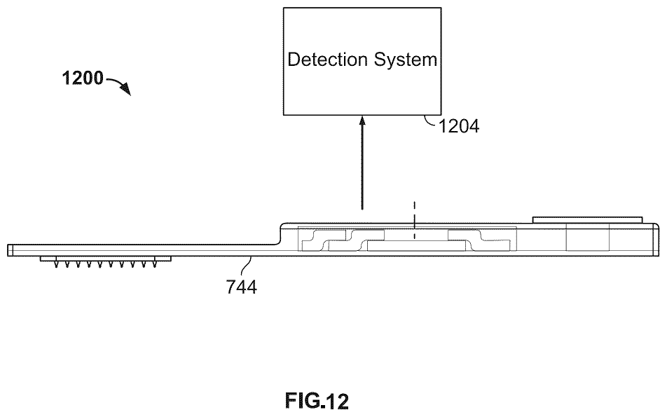

Referring now to FIG. 12, a detection kit 1200 is presented, illustrating the integration of device casing removal mechanisms with external detection systems for biomolecule level analysis. Detection kit 1200 may include an imaging chamber, optical sensors, processing electronics, and user interface components designed to quantify biomolecule levels based on visual indications generated by sensing strips within housing 744. The detection system 1204 may be configured to detect insertion of the microfluidic system through physical sensors, optical sensors, or mechanical alignment features that recognize when the device components are properly positioned for analysis. Detection system 1204 may incorporate automated positioning mechanisms, calibration systems, and image capture capabilities that interface with the transparent housing of the microfluidic system to perform colorimetric analysis, fluorescence detection, or other optical measurement techniques. The detection kit 1200 configuration enables comprehensive biomolecule monitoring workflows by combining the wearable microfluidic device 100 with external detection capabilities, providing quantitative biomolecule concentration measurements, temporal trend analysis, and diagnostic outputs through integrated software applications or mobile device interfaces.

Housing 744 may be inserted into detection system 1204 through various mechanical interface mechanisms designed to ensure proper alignment and secure positioning for accurate analysis. The insertion process may utilize sliding mechanisms where housing 744 slides linearly into a detection chamber or slot within detection system 1204 along predetermined guide rails, tracks, or channels that maintain consistent positioning relative to optical sensors and imaging components. Insertion forces may range from about 2 N to about 15 N applied in directions parallel to the sliding axis, with mechanical stops or detents providing tactile feedback when proper insertion depth is achieved. Alternative insertion mechanisms may include snap-fit connections where mechanical features such as flexible tabs, spring-loaded latches, or cam mechanisms secure housing 744 within detection system 1204, requiring activation forces ranging from about 1 N to about 10 N for engagement and release. Magnetic coupling systems may utilize permanent magnets or electromagnets embedded within both housing 744 and detection system 1204 to provide automatic alignment and secure retention during analysis, with magnetic forces ranging from about 5 N to about 25 N. The insertion interface may incorporate standardized dimensions and form factors that enable compatibility across multiple detection system platforms, including industry-standard microplate formats, custom cartridge dimensions, or proprietary form factors optimized for specific biomolecule detection applications.

Detection system 1204 may incorporate various sensing and analysis technologies to detect and quantify biomolecule levels based on visual, optical, or electrical signals generated by the assay system components within housing 744. The detection process may begin with automated recognition of housing 744 insertion through physical sensors, optical sensors, proximity sensors, or mechanical switches that confirm proper positioning and initiate analysis protocols. Detection system 1204 may include illumination sources such as light-emitting diodes (LEDs), halogen lamps, or laser diodes that provide controlled lighting conditions for colorimetric analysis, with illumination wavelengths ranging from about 400 nm to about 700 nm for visible light detection or about 280 nm to about 400 nm for ultraviolet fluorescence excitation. Imaging components may comprise high-resolution cameras with resolutions ranging from about 1 megapixels to about 108 megapixels, charge-coupled device (CCD) sensors, complementary metal-oxide-semiconductor (CMOS) sensors, or specialized optical detectors optimized for specific detection modalities including colorimetric, fluorescent, chemiluminescent, or electrochemical measurements. Detection system 1204 may perform automated image capture of sensing strips within the assay system, utilizing computer vision algorithms, machine learning models, or artificial intelligence systems to identify test zones, control zones, and reaction areas where biomolecule detection occurs. The detection process may include color analysis algorithms that quantify color intensity, hue, saturation, or color transitions corresponding to biomolecule concentration levels, with measurement precision enabling detection of biomolecule concentrations ranging from about 0.1 ng/dL to about 1000 ng/dL depending on the specific biomolecule being analyzed.

The output of detection system 1204 may include comprehensive biomolecule analysis results presented in various formats designed to facilitate clinical interpretation and patient monitoring. Quantitative outputs may provide precise biomolecule concentration measurements expressed in clinically relevant units including nanograms per deciliter (ng/dL), picograms per milliliter (pg/mL), international units per liter (IU/L), or other standardized concentration units depending on the specific biomolecule being measured. Detection system 1204 may generate numerical readouts with measurement ranges spanning from about 0.1 ng/dL to about 1000 ng/dL for biomolecules such as testosterone, estradiol, progesterone, cortisol, luteinizing hormones, follicle stimulating hormones, or thyroid hormones, with measurement precision and accuracy optimized for clinical decision-making applications. Qualitative outputs may include categorical determinations such as “detected/not detected,” “normal/abnormal,” or “low/normal/high” classifications based on established reference ranges for specific patient demographics, age groups, or clinical conditions. The detection system may provide temporal trend analysis by tracking biomolecule level changes over time periods ranging from minutes to months, generating graphical displays, statistical summaries, or predictive models that identify patterns in hormonal fluctuations and enable monitoring of treatment responses or disease progression. Output formats may include digital displays on integrated screens, printed reports for clinical documentation, mobile application interfaces for patient access, cloud-based dashboards for healthcare provider monitoring, or electronic health record integration formats that facilitate seamless incorporation into existing clinical workflows and enable longitudinal patient care coordination.

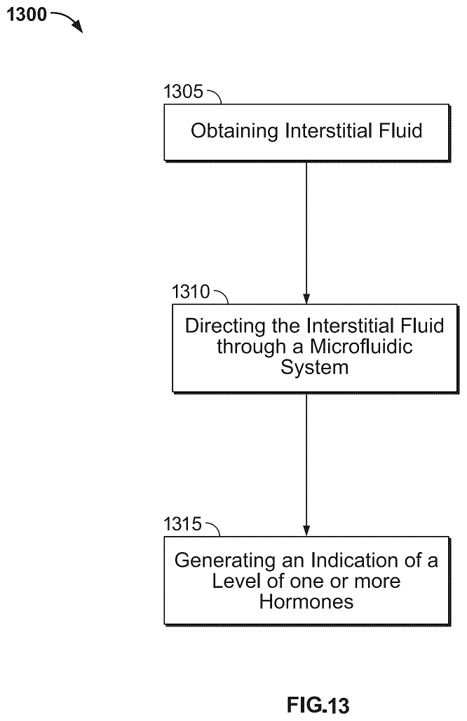

Referring now to FIG. 13, a method of generating an indication of a level of one or more biomolecules using a device includes step 1305, in which interstitial fluid is obtained. Interstitial fluid may be obtained from a microneedle array of a device. For instance, the microneedle array may include one or more microneedles that may pierce into a skin of a user and may pull interstitial fluid from the user.

At step 1310, method 1300 includes directing the interstitial fluid through a microfluidic system of a device. Interstitial fluid may be directed through a microfluidic system via one or more fluidic channels. For instance, fluidic channels may be in fluidic communication with outlets of one or more microneedles and may allow for a flow of fluid through the one or more microneedles into a microfluidic system. A microfluidic system may have a first channel that may regulate fluid flow, a fluidic valve coupled to the first channel, and a second channel coupled to the fluidic valve. A second channel may have a debonding agent that may debond biomolecules from other molecules.

At step 1315, method 1300 includes generating an indication of a level of one or more biomolecules. An indication of a level of one or more biomolecules may be generated through an assay system of a device. An assay system may include one or more sensing strips configured to generate an indication of a level of one or more biomolecules in a fluid of an individual.

Any step of method 1300 may be performed as described above with reference to FIGS. 1-12, without limitation.

In some embodiments, operation of devices described herein may include the following steps: (1) unpacking the device from packaging; (1.5) optionally wiping a portion of their skin with a cleaning solution; (2) removing a protective film backing and placing the device on a user's upper arm with applied pressure for strong adhesive contact; (3) activating the device by pressing or twisting needle inserter 116 to deploy microneedle array 132 into the skin, then pressing pressure actuator 112 to initiate fluid extraction; (4) allowing the device to collect interstitial fluid during normal user activities for a predetermined time period ranging from about 10 minutes to about 30 minutes; (4.5) optionally activating a heat applier to warm the skin surface to temperatures ranging from about 35° C. to about 45° C. to enhance biomolecule diffusion from blood to interstitial fluid; (5) removing the device from the user's arm and extracting the assay system components through access ports in housing 744; (6) inserting the removed assay system components into an external imaging device for analysis; and (7) viewing biomolecule level results through a connected mobile application or display system.

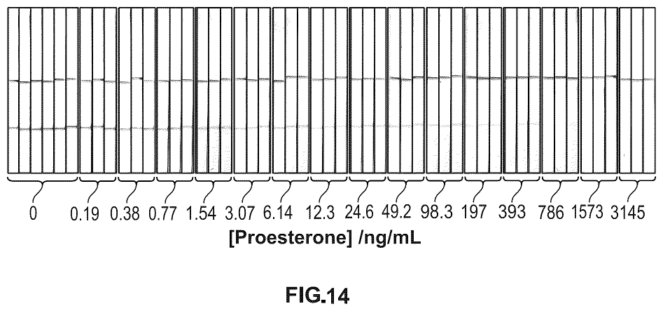

FIG. 14 illustrates a plurality of sensing strips for detection in phosphate buffered saline (PBS) buffer. In particular, FIG. 14 displays antibody-based competitive lateral flow assay test strips spanning a full concentration range of progesterone from 3145 ng/ml down to 0.19 ng/mL. On the left-hand side, six replicates of the negative control (0 ng/ml progesterone) are displayed, showing strong test lines as expected in the absence of target biomolecule. Moving from left to right, increasing concentrations of progesterone result in progressively weaker test lines, consistent with the competitive assay format where target biomolecules compete for binding with labeled affinity binding agents. At the highest concentrations on the right side, the test zone exhibits faint or absent signal intensity, while the control zone remains visible across all strips, confirming assay validity and proper lateral flow function.

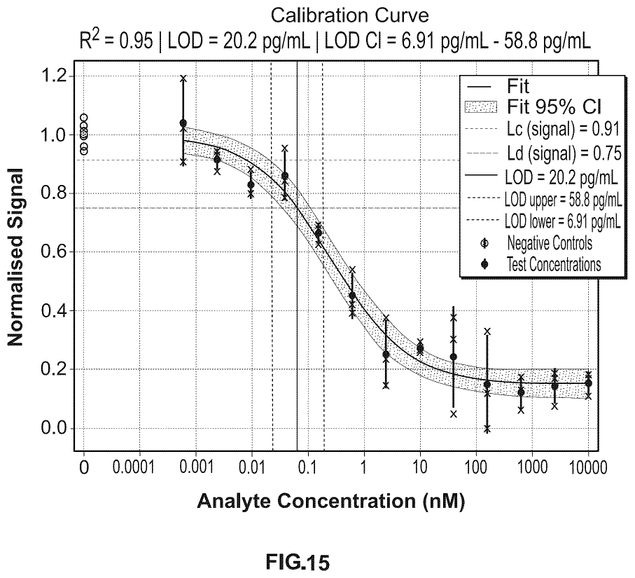

FIG. 15 shows a calibration curve for progesterone detection using sensing strip readouts, demonstrating the relationship between progesterone concentration (ranging from 0 to 3145 ng/ml) and signal response. The calibration curve shows an R² value of 0.95, indicating strong correlation between concentration and signal. The limit of detection (LOD) is determined to be 20.2 pg/mL with a confidence interval ranging from 6.91 pg/mL to 58.8 pg/mL. The calibration curve includes both negative controls and test concentrations, with fit lines and 95% confidence intervals shown to validate the assay performance and quantitative capabilities of the sensing strips.

### Additional Embodiments

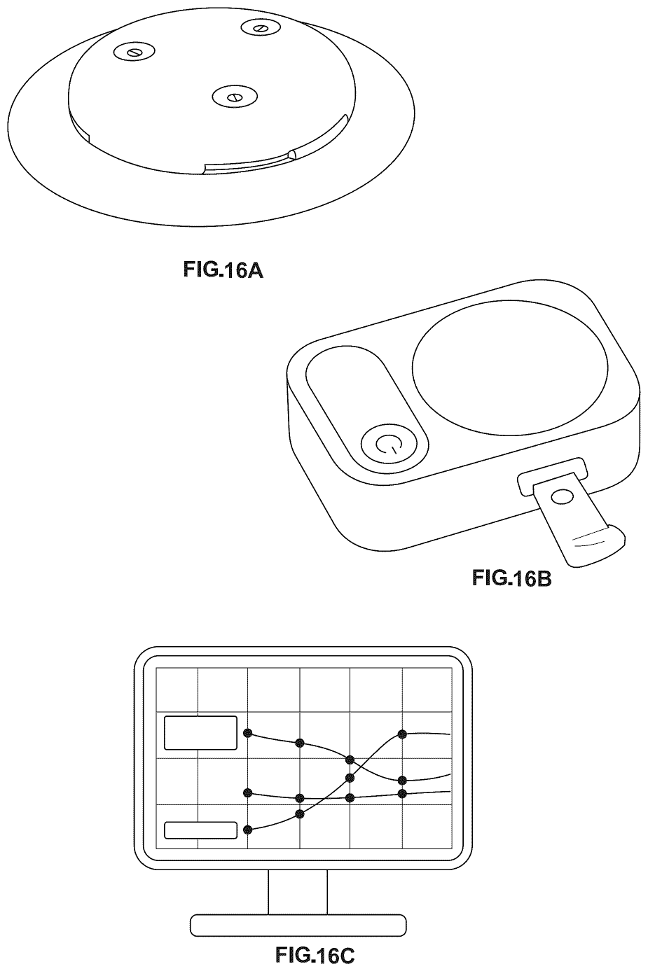

FIGS. 16A-16C are perspective views including (FIG. 16A) a device for intermittent biomolecule detection, (FIG. 16B) a second device that allows the sensing strip to be read, and (FIG. 16C) a computer readout depicting the relative levels of three different biomolecules.

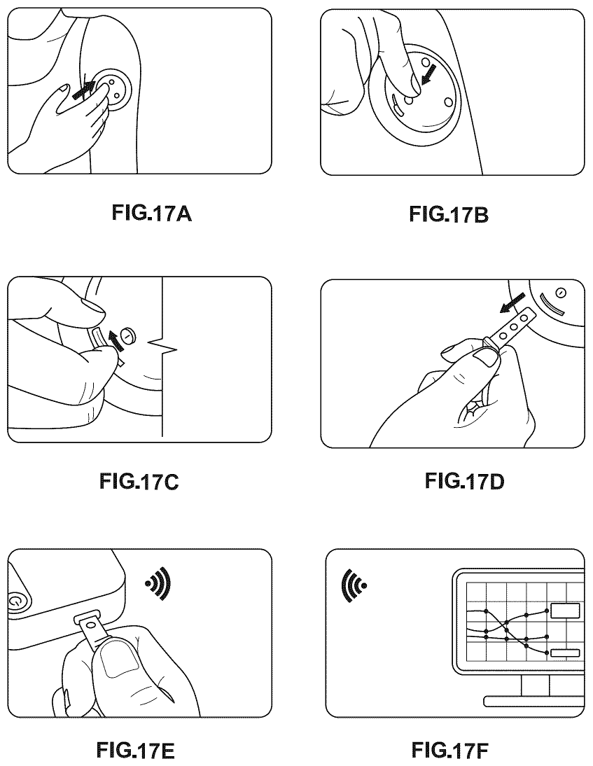

FIGS. 17A-17F show a process by which the device works with FIG. 17A showing the individual (patient) placing the microneedle device on the arm, FIG. 17B showing an individual depressing an on/off button, FIG. 17C showing the individual rotating the slider allowing the solid blocking component to be deactivated and allowing the sensing strip attachment to record one or more biomolecule concentration levels, FIG. 17D showing removal of the sensing strip (e.g., a paper strip), FIG. 17E showing the paper strip being inserted into a second device that reads the sensing strip, and FIG. 17F showing a computer readout of the various levels of biomolecules, which optionally works by wifi.

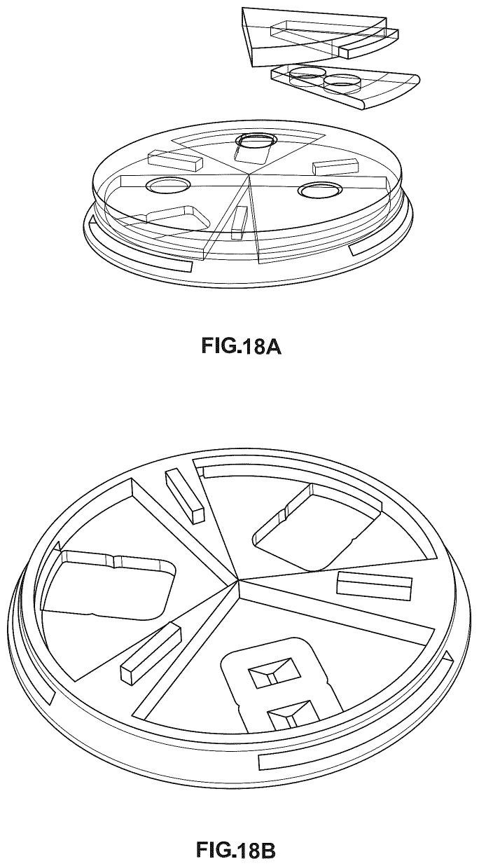

FIGS. 18A-18B show perspective views of the microfluidic chamber of the device of the present disclosure with FIG. 18A showing the components of the device, which includes sensing chamber and the strip holder and sample strip, and FIG. 18B showing a top down view and the inner view of the microfluidic chamber, which includes a volume chamber that is prefilled with 3 μL of phosphate buffered saline (PBS).

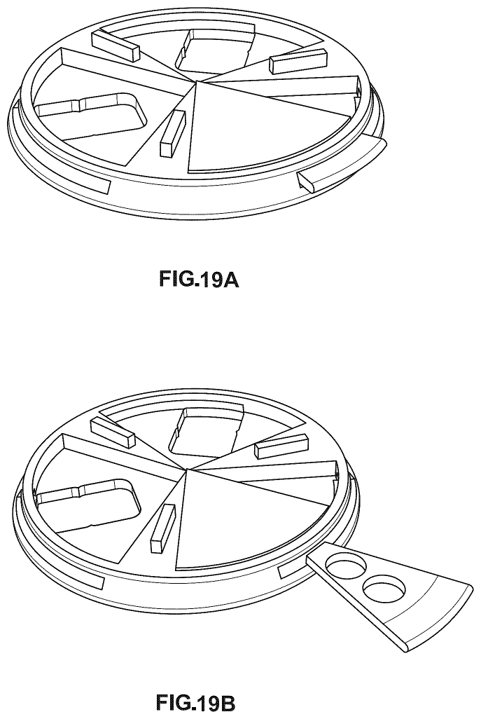

FIGS. 19A-19B show perspective views of the microfluidic chamber of the device of the present invention with FIG. 19A showing the device, and FIG. 19B showing a top down view of the microfluidic chamber and the place of insertion for the sample strip.

In a variation, the one or more aptamers include single stranded DNA. In a variation, the one or more aptamers comprise RNA. In a variation, the one or more aptamers comprise DNA and RNA. In a variation, the one or more biomolecules cause a conformational change in the one or more aptamers, thereby generating an electrical signal that is measured by one or more sensors. In a variation, the biomolecule is a sex hormone. In a variation, the sex hormone is estrogen, progesterone, luteinizing hormone, dihydrotestosterone, testosterone, cortisol, hydrocortisone, follicle stimulating hormone, melatonin, and/or Anti-Müllerian hormone (AMH).

In a variation, the device further includes one or more of a sensing chamber, or a slider mechanism, the slider mechanism comprising one or more of a solid blocking component, and/or a sensing strip attachment. In a variation, the device includes both a sensing chamber and a slider mechanism. In a variation, the sensing chamber comprises a buffer solution, the sensing chamber configured to allow the interaction of the one or more biomolecules with the one or more aptamers, the slider mechanism configured so that the solid blocking component blocks the sensing chamber from interaction with the one or more biomolecules until the on/off button is depressed. In a variation, the sensing strip attachment records information from the one or more sensors. In a variation, the sensing strip attachment comprises paper, the paper being removable. In a variation, the sensing strip attachment is removable and can be read and/or interpreted by a second device. In a variation, the one or more aptamers comprise DNA, and the DNA is configured to specifically bind the one or more biomolecules, the one or more biomolecules comprising estradiol (E2), progesterone (P4), testosterone (T), and/or luteinizing hormone (LH). In a variation, polymerase chain reaction (PCR) is used to generate the DNA. In a variation, the one or more sensors sample the electrical signal at intervals between 10 seconds to an hour.

In an embodiment, the present disclosure relates to a method of measuring a biomolecule concentration in an individual, the method including administering a device adapted and configured for ascertaining the biomolecule concentration to the individual, the device including a microneedle array patch, the device having an on/off button that allows for biomolecule monitoring, the device designed and configured to sample interstitial fluid space, the device including one or more aptamers that allow for binding of one or more biomolecules from the interstitial space, the one or more biomolecules having a correlation with a concentration level of the biomolecule being assayed. In a variation, the one or more aptamers are DNA or RNA, or both. In a variation, the on/off button is operationally connected to a sensing chamber, and/or a slider mechanism, the slider mechanism including one or more of a solid blocking component, and/or a sensing strip attachment. In a variation, the sensing chamber is configured to allow the interaction of the one or more biomolecules with the one or more aptamers, the slider mechanism configured so that the solid blocking component blocks the sensing chamber from interaction with the one or more biomolecules until the on/off button is depressed. In a variation, the sensing strip attachment is removable and can be read and/or interpreted by a second device.

In an embodiment, the present disclosure relates to a device as enumerated above that can further administer one or more biomolecules to the interstitial fluid space.

In an embodiment, the device and method involves generating a microneedle patch with the sensors and the needles as described herein, attaching them to an individual (patient) wherein the patch microneedles are able to access the interstitial fluid space, having the individual turn on the microneedle patch using the on/off button and allowing the patch to measure the biomolecule.

It should be understood and it is contemplated and within the scope of the present disclosure that any feature that is enumerated above can be combined with any other feature that is enumerated above as long as those features are not incompatible. Whenever ranges are mentioned, any real number that fits within the range of that range is contemplated as an endpoint to generate subranges. In any event, the disclosure is defined by the below claims.

What is claimed is:

1. A device for intermittent monitoring of a biomolecule in a human body, the device comprising:
a device casing;
a microneedle array comprising an array of needles configured to extract interstitial fluid from a user;
a microfluidic system defining a fluidic channel comprising a biomolecule debonding agent and in fluidic communication with the microneedle array and is configured to facilitate flow of the extracted interstitial fluid therethrough; and
an assay system in fluidic communication with the microfluidic system, the assay system comprising an affinity binding agent capable of generating a quantitative indication of a level of one or more biomolecules present in the extracted interstitial fluid,
wherein the microneedle array, the microfluidic system, and the assay system are disposed within the device casing.

2. The device of claim 1, further comprising a pressure actuator configured to provide a pressure to the microfluidic system.

3. The device of claim 1, wherein the biomolecule debonding agent is disposed in a coating.

4. The device of claim 1, wherein the biomolecule debonding agent is disposed in a blister pack.

5. The device of claim 1, wherein the assay system is removably couplable to the device casing.

6. The device of claim 1, wherein each needle of the array of needles has a length between about 300 μm to about 1500 μm.

7. The device of claim 1, wherein a tip of each needle of the array of needles is angled at an angle of about 10 degrees to about 60 degrees.

8. The device of claim 1, wherein each needle of the microneedle array is spaced apart by about 500 μm to about 5,000 μm.

9. The device of claim 1, wherein the microneedle array comprises a rib designed to create positive pressure on a surface of skin of the user.

10. The device of claim 9, wherein the positive pressure is from a range of about 10 kPA to about 100 kPa.

11. The device of claim 1, wherein the microfluidic channel comprises a metering zone.

12. The device of claim 1, wherein the microfluidic system comprises a flexible membrane for inducing flow of the extracted interstitial fluid.

13. The device of claim 1, wherein the microfluidic system comprises a fluid actuator for controlling flow of the extracted interstitial fluid.

14. The device of claim 1, wherein the flow of the extracted interstitial fluid occurs through capillary pressure induced by a surface of the fluidic channel.

15. The device of claim 1, wherein the assay system comprises a lateral flow assay.

16. The device of claim 1, wherein the affinity binding agent is disposed in a porous strip.

17. The device of claim 1, further comprising an adhesive surface designed to adhere the device to a portion of the user.

18. The device of claim 1, wherein at least one microneedle of the microneedle array defines two or more holes.

19. The device of claim 1, wherein the biomolecule debonding agent is one of a chemical, enzymatic, or biological agent.

20. A biomolecule detection kit comprising:
a device comprising:
a device casing;
a microneedle array comprising an array of needles configured to extract interstitial fluid from a user;
a microfluidic system defining a fluidic channel comprising a biomolecule debonding agent, wherein the fluidic channel is in fluidic communication with the microneedle array and is configured to facilitate flow of the extracted interstitial fluid therethrough; and
an assay system in fluidic communication with the microfluidic system, the assay system comprising an affinity binding agent capable of generating an indication of a level of one or more biomolecules present in the extracted interstitial fluid,
wherein the microneedle array, the microfluidic system, and the assay system are disposed within the device casing; and
a detection system configured to detect biomolecule levels based on the indication.

21. The biomolecule detection kit of claim 20, wherein the detection system comprises a camera for capturing an image of the assay system for colorimetric analysis.

22. The biomolecule detection kit of claim 20, wherein the detection system is configured to detect at least one of fluorescence and chemiluminescence of the assay system.

23. A method of intermittent monitoring of a biomolecule in a human body using a microfluidic device, the method comprising:
obtaining, from a microneedle array of the microfluidic device, interstitial fluid of a user;
directing, through a fluidic channel of a microfluidic system of the microfluidic device, the interstitial fluid to an assay system of the microfluidic device; and
generating, through the assay system, an indication of a level of one or more biomolecules present in the extracted interstitial fluid.

24. A device for intermittent monitoring of a biomolecule in a human body, the device comprising:
a device casing;
a microneedle array comprising an array of needles configured to extract interstitial fluid from a user;
a microfluidic system defining a fluidic channel in fluidic communication with the microneedle array and configured to facilitate flow of the extracted interstitial fluid therethrough; and
an assay system in fluidic communication with the microfluidic system, the assay system comprising:
an affinity binding agent capable of generating a quantitative indication of a level of one or more biomolecules present in the extracted interstitial fluid; and
a slider mechanism configured to fluidically couple the assay system with the microfluidic system, thereby allowing the interstitial fluid to flow into the assay system; and
wherein the microneedle array, the microfluidic system, and assay system are disposed within the device casing.
